(19) United States
(12) Patent Application Publication
(10) Pub. No.: US 2017/0127911 A1
(43) Pub. Date: May 11, 2017

(54) MASTER - SLAVE FLEXIBLE ROBOTIC ENDOSCOPY SYSTEM

(71) Applicants: ENDOMASTER PTE LTD, Singapore (SG); HOYA CORPORATION, Tokyo (JP)

(72) Inventors: Tomonori Yamamoto, Singapore (SG); Isaac David Penny, Singapore (SG); Christopher Lee Shih Hao Sam Soon, Singapore (SG); Hoang-Ha Tran, Singapore (SG); Tae Zar Lwin, Singapore (SG); Tsun En Tan, Singapore (SG); Naoyuki Naito, Tokyo (JP); Takahiro Kobayashi, Tokyo (JP); Makio Oishi, Tokyo (JP)

(73) Assignees: Endomaster PTE LTD, Singapore (SG); Hoya Corporation, Tokyo (JP)

(21) Appl. No.: 15/127,397

(22) PCT Filed: Mar. 19, 2015
(86) PCT No.: PCT/SG2015/050044

§ 371 Date: Sep. 19, 2016

Related U.S. Application Data
(60) Provisional application No. 61/955,232, filed on Mar. 19, 2014.

(51) Int. Cl. A61B 1/005; A61B 34/37; A61B 34/00; A61B 1/00

(52) U.S. Cl. CPC A61B 1/0053; A61B 1/00133; A61B 34/37; A61B 34/71; A61B 2034/715; A61B 2034/301

(57) ABSTRACT

A flexible robotic endoscopy slave system includes an endoscope body and a flexible elongate shaft extending therefrom into which at least one tendon driven robotic endoscopic instrument is insertable; a docking station with which the endoscope body is releasably dockable; and a translation mechanism for selectively longitudinally displacing the endoscopic instrument(s) within the flexible elongate shaft when the endoscope body is docked. The translation mechanism can carry and selectively displace actuators that drive each robotic endoscopic instrument by way of tendons. At least one degree of freedom (DOF) of robotic instrument motion is controlled by a pair of actuators and a corresponding pair of tendons. Actuation engagement structures releasably couple the actuators to an adapter structure for driving each endoscopic instrument. Tendon pretensioning can occur automatically under programmable control. A roll joint without tendon crimping structures can be employed in a robotic endoscopic instrument for reducing tendon wear and roll joint spatial volume.

### TECHNICAL FIELD

A slave system of a master-slave flexible robotic endoscopy system includes an endoscope body and a flexible elongate shaft extending therefrom into which at least one tendon driven robotic endoscopic instrument is insertable; a docking station with which the endoscope body is releasably dockable; and a translation mechanism operable to selectively longitudinally displace the endoscopic instrument(s) within the flexible elongate shaft when the endoscope body is docked. Actuation engagement structures releasably couple motorbox actuators to an adapter structure for driving each endoscopic instrument. For at least some degrees of freedom (DOF) of spatial motion, two actuators and two corresponding tendons can control instrument motion per DOF. Tendon pretensioning can occur automatically under programmable control. A roll joint without tendon crimping structures can be employed in a robotic endoscopic instrument for reducing tendon wear and roll joint spatial volume.

### BACKGROUND

Multiple master-slave flexible robotic endoscopy systems have been proposed or are currently in development. For instance, International Patent Application No. PCT/SG2013/000408 and International Patent Publication No. WO 2010/138083 describe master-slave flexible robotic endoscopy systems in which tendon-driven robotic arms and corresponding end effectors are insertable into an endoscope body having a flexible elongate shaft extending therefrom, such that the robotic arms and end effectors can extend beyond a distal end of the flexible elongate shaft for performing an endoscopy procedure. The tendons that drive the robotic arms and their end effectors reside in sheath structures, such as helical coil sheaths.

Portions of a flexible robotic endoscopy system, including a flexible elongate shaft that carries robotic arms and corresponding end effectors, which are intended for insertion into the human body require size minimization. Unfortunately, body-insertable portions of some existing flexible robotic endoscopy systems have larger diameters or cross-sectional areas than desired relative to internal bodily environments in which they are intended to be deployed.

During an endoscopy procedure, the robotic arms and end effectors carried by the flexible elongate shaft must be precisely manipulable at all times in response to control signals generated by a surgeon. The flexibility provided by a flexible robotic endoscopy system offers the promise of insertion of the flexible elongate shaft into a natural body orifice, followed by routing of the flexible elongate shaft along a tortuous or highly tortuous path to a target site at which the surgeon can perform the endoscopy procedure. However, such flexibility itself gives rise to difficulties with respect to ensuring that the robotic arms and their end effectors remain precisely controllable regardless of the tortuosity of the path along which the flexible elongate shaft is routed. More particularly, the tensions of tendons by which the robotic arms and end effectors are spatially manipulated can vary significantly depending upon the path along which tendons are routed, resulting in tendon slack or tendon backlash that degrades consistent, high precision controllability of the robotic arms and their end effectors.

A need exists for a flexible robotic endoscopy system that overcomes such problems.

### SUMMARY

In accordance with an aspect of the present disclosure, a master-slave endoscopy system includes: an endoscope having a main body from which a flexible elongate shaft extends, the flexible elongate shaft spanning a length between a proximal end and a distal end thereof, the flexible elongate shaft having a plurality of channels disposed therein along its length including a first channel, a second channel, and a third channel; a robotically driven actuation assembly removably inserted into the first channel, the robotically driven actuation assembly including a robotic arm having a robotically driven end effector coupled thereto, and a second plurality of tendons operable for spatially manipulating the robotic arm and its end effector in response to forces applied thereto; an imaging endoscope removably inserted into the second channel; and a manually driven actuation assembly removably inserted into the third channel, the manually driven actuation assembly having a manually operated endoscopic instrument coupled thereto.

The first set of actuators is couplable to the robotically driven actuation assembly, and is configured for applying forces to the second plurality of tendons thereof.

The imaging endoscope can form a portion of an imaging endoscope assembly including an adapter by which the imaging endoscope is couplable to an actuator configured for providing surge displacement to the imaging endoscope. The imaging endoscope assembly can further include a plurality of tendons carried therein coupled by way of the adapter to a second set of actuators configured for providing the imaging endoscope with at least one of heave, sway, and pitch motion.

The robotically driven actuation assembly further includes an adapter removably couplable to the first set of actuators, and is configured for motion in accordance with a predetermined number of degrees of freedom (DOF), where the first set of actuators includes two actuators corresponding to at least one DOF.

In accordance with an aspect of the present disclosure, a master-slave endoscopy system includes: (a) an endoscope having a main body from which a flexible elongate shaft extends, the flexible elongate shaft spanning a length between a proximal end and a distal end thereof, the flexible elongate shaft having a set of channels disposed therein along its length into which a set of actuation assemblies are insertable, the plurality of channels including a first channel and a second channel; (b) a set of flexible robotically driven actuation assemblies carried by the set of channels, each robotically driven actuation assembly including: a robotic arm having a robotically driven end effector coupled thereto; and a plurality of tendons coupled to the robotic arm and configured for controlling motion of the robotic arm and its end effector in accordance with a predetermined number of degrees of freedom (DOF), wherein two tendons control each DOF of the robotic arm; (c) a set of actuators corresponding to each robotically driven actuation assembly, each actuator controllable by way of a set of input devices with which a surgeon can interact, each actuator configured for selectively applying torque to a tendon of its corresponding robotically driven actuation assembly in response to surgeon input directed to the set of input devices, wherein two actuators control each DOF of the robotic arm; and (d) a processing unit configured for performing a tendon pretensioning or retensioning procedure to automatically establish a level of tension in the plurality of tendons of each robotically driven actuation assembly by way of: (i) applying torque to each actuator of the robotically driven actuation assembly in accordance with stored torque parameters associated with a representative tortuosity configuration that is expected to correspond to a tortuosity of a path along which the robotically driven actuation assembly is routed; or (ii) for each tendon of the robotically driven actuation assembly: dynamically determining a torque transition point between a slack condition and a no-slack condition of the tendon, and applying torque to an actuator corresponding to the tendon (e.g., an actuator to which the tendon is secured or attached) at a torque level defined by the torque transition point determined therefor.

Applying torque to each tendon of the robotically driven actuation assembly in accordance with stored torque parameters associated the representative tortuosity configuration can be performed outside of an operating theater prior to performance of an endoscopic procedure, or after insertion of each robotically driven actuation assembly into a channel of the flexible elongate shaft.

Dynamically determining for each tendon the torque transition point between the slack condition and the no-slack condition can occur immediately prior to or during performance of an endoscopic procedure. Dynamically determining for each tendon the torque transition point between the slack condition and the no-slack condition can include: determining or measuring a tendon tension profile corresponding to the tendon; and calculating a first and/or a second derivative of the tendon tension profile.

The system can further include an instrument adapter corresponding to each robotically driven actuation assembly, the instrument adapter removably couplable to the set of actuators for selectively coupling the plurality of tendons robotically driven actuation assembly to the set of actuators, wherein the instrument adapter is configured for maintaining tension applied to each tendon of the robotically driven actuation assembly when decoupled from the set of actuators.

In accordance with an aspect of the present disclosure, a master-slave endoscopy system includes: (a) a set of robotically driven actuation assemblies, each robotically driven actuation assembly having: a robotic arm having a robotically driven end effector coupled thereto; and a plurality of tendons configured for controlling motion of the robotic arm and the end effector in accordance with a predetermined number of degrees of freedom (DOF); and (b) an instrument adapter corresponding to each robotically driven actuation assembly and coupled to the tendons thereof, the instrument adapter couplable to a set of mechanical elements for selectively coupling the plurality of tendons of the robotically driven actuation assembly to a set of actuators, the instrument adapter including: (i) a rotatable shaft corresponding to each tendon of the robotically driven actuation assembly, the rotatable shaft having a longitudinal axis relative to which the tendon is circumferentially wound; and (ii) a first tension maintenance element and a second tension maintenance element corresponding to each rotatable shaft, wherein the first tension maintenance element is displaceable relative to the second tension maintenance element for selective engagement with and disengagement from the second ratchet element, and wherein the first tension maintenance element is configured for mating engagement with the second tension maintenance element when the instrument adapter is decoupled from the set of mechanical elements to prevent rotation of the shaft and thereby maintain a level of tension in the tendon. The first and second tension maintenance elements can each include or be one of a ratchet element and a friction plate.

The instrument adapter can further include a resilient biasing element that maintains the first tension maintenance element and the second tension maintenance element in an engaged state when the instrument adapter is decoupled from the set of mechanical elements. The resilient biasing element can be displaceable relative to the shaft for disengaging the first tension maintenance element from the second tension maintenance element when the instrument adapter is coupled to the set of mechanical elements such that the shaft is rotatable.

The set of actuators can include two actuators corresponding to each DOF, wherein for each DOF the instrument adapter includes a first rotatable shaft relative to which a first tendon is circumferentially wound and a second rotatable shaft relative to which a second tendon is circumferentially wound for controlling motion of the robotic arm and end effector of the robotically driven actuation assembly.

In accordance with an aspect of the present disclosure, a master-slave endoscopy system includes: (a) an endoscope having a main body from which a flexible elongate shaft extends, the flexible elongate shaft spanning a length between a proximal end and a distal end thereof, the flexible elongate shaft having a set of channels disposed therein along its length into which a set of actuation assemblies are insertable, the plurality of channels including a first channel and a second channel; (b) a set of robotically driven actuation assemblies, each robotically driven actuation assembly including: a robotic arm having a robotically driven end effector coupled thereto; a plurality of tendons coupled to the robotic arm and configured for controlling motion of the robotic arm and the end effector in accordance with a predetermined number of degrees of freedom (DOF); and an outer sleeve surrounding the plurality of tendons; (c) a first instrument adapter corresponding to each robotically driven actuation assembly and coupled to the tendons thereof, the first instrument adapter couplable to a set of mechanical elements for selectively coupling the plurality of tendons of the robotically driven actuation assembly to a set of robotic arm/end effector manipulation actuators; and (d) a translation mechanism configured for independently translating each robotically driven actuation assembly along a predetermined fraction of the length of the flexible elongate shaft to effectuate surge displacement of the robotically driven actuation assembly, the translation mechanism comprising one of: (i) a collar carried by each outer sleeve of the set of robotically driven actuation assemblies; and a translation unit including: a receiver configured for matingly receiving an outer sleeve of a robotically driven actuation assembly, a linear actuator corresponding to each receiver and configured for selectively translating the receiver along the predetermined fraction of the flexible elongate shaft's length; (ii) a second instrument adapter to which each first instrument adapter is matingly engageable for coupling the tendons of the robotically driven actuation assembly corresponding to the first instrument adapter to the set of robotic arm/end effector manipulation actuators; and a translation unit configured for carrying each first instrument adapter as well as a second instrument adapter matingly engageable therewith, and displacing each first instrument adapter and each second instrument adapter that are matingly engaged to effectuate surge displacement of individual robotically driven actuation assemblies along the predetermined fraction of the flexible elongate shaft's length; and (iii) a translation unit configured for displacing individual sets of robotic arm/end effector manipulation actuators and each first instrument adapter coupled thereto to effectuate surge displacement of individual robotically driven actuation assemblies along the predetermined fraction of the flexible elongate shaft's length.

Each second instrument adapter cam be coupled to the set of robotic arm/end effector manipulation actuators by a tether having a plurality of tendons therein.

The system further includes a docking station to which a portion of the main body of the endoscope is detachabley engageable. The translation mechanism can be carried by the docking station; and a patient side cart can carry the docking station.

The system can further include a set of cradle structure or cradles carrying the translation mechanism, wherein each cradle of the set of cradles corresponds to an individual robotically driven actuation assembly, and each cradle of the set of cradles is coupled to a roll motion actuator configured for individually rotating the cradle and its corresponding robotically driven actuation assembly about a roll axis to provide roll motion to the robotic arm and end effector of the robotically driven actuation assembly. A docking station to which a portion of the main body of the endoscope is detachably engageable can carry the translation mechanism and the set of cradles.

In accordance with an aspect of the present disclosure, a tendon controlled robotic arm includes: a roll joint including a drum structure having a central axis therethrough, the roll joint configured for rotating portions of the robotic arm about the central axis in response to actuation of a tendon coupled carried thereby, the roll joint excluding tendon crimp terminations thereon for anchoring a tendon to the roll joint.

The drum structure includes an outer surface, and the roll joint can include: a clockwise actuation pulley carried by the outer surface and having a channel through which a clockwise actuation tendon extends for rotating the roll joint in a clockwise direction; and a counterclockwise actuation pulley carried by the outer surface and having a channel through which a counterclockwise actuation tendon extends for rotating the roll joint in a counterclockwise direction.

The drum structure can include at least one omega shaped or U-shaped segment that respectively provides a corresponding omega shaped or U-shaped channel, passage, or groove through which a tendon for controlling rotation of the roll joint is routable.

A set of eyelets can be formed in the drum, through which a tendon is routable such that the tendon is disposed on each of an outer surface of the drum and an inner surface of the drum. The drum structure can carry a tendon along a tendon routing path from an outer side of the drum, into and through a thickness of the drum to an inner side of the drum, and back through the thickness of the drum to the outer side of the drum. An adhesive can secure an outer surface of a tendon to portions of the drum.

### BRIEF DESCRIPTION OF THE DRAWINGS

FIGS. 1A and 1B are schematic illustrations of a master-slave flexible robotic endoscopy system in accordance with an embodiment of the disclosure.

FIG. 2 is a schematic illustration of a master system in accordance with an embodiment of the present disclosure.

FIG. 3 is a schematic illustration of a slave system in accordance with an embodiment of the present disclosure.

FIGS. 4A-4D are schematic illustrations of a representative transport endoscope, first and second actuation assemblies, and an imaging endoscope assembly, respectively, in accordance with an embodiment of the present disclosure.

FIG. 5 is a schematic illustration of a pair of robotic arms and corresponding end effectors carried thereby, as well as an imaging endoscope, positioned in an environment beyond a distal end of a transport endoscope in accordance with an embodiment of the present disclosure.

FIG. 6A is a representative cross sectional illustration of a transport endoscope shaft in accordance with an embodiment of the present disclosure.

FIG. 6B is a representative cross sectional illustration of a transport endoscope shaft in accordance with another embodiment of the present disclosure.

FIGS. 7A-7C are schematic illustrations showing imaging endoscope assembly insertion into a transport endoscope, imaging connector assembly coupling to an imaging subsystem, imaging input adapter coupling to an imaging output adapter of a motorbox, and an endoscopy support function connector assembly coupling to a valve control unit in accordance with an embodiment of the present disclosure.

FIGS. 8A-8B are schematic illustrations showing transport endoscope docking to a docking station, with portions of outer sleeves/coils of actuation assemblies and an outer sleeve of an imaging endoscope assembly inserted into the transported endoscope, and such outer sleeves securely coupled to a translation unit of the docking station.

FIG. 8C is a schematic illustration showing a representative translation unit carried by the docking station, and a representative manner in which collar elements corresponding to actuation assemblies and an imaging endoscope assembly are retained by the translation unit.

FIG. 9 is a schematic illustration showing coupling of an instrument input adapter of each actuation assembly to a corresponding instrument output adapter corresponding to a motorbox in accordance with an embodiment of the present disclosure.

FIG. 10 is a perspective cutaway view showing representative internal portions of an instrument input adapter mounted to an instrument output adapter of the motorbox in accordance with an embodiment of the present disclosure.

FIG. 11 is a corresponding cross sectional illustration showing representative internal portions of the instrument adapter and instrument output adapter when coupled together or matingly engaged in accordance with an embodiment of the present disclosure.

FIGS. 12A-12D are cross sectional illustrations showing representative internal portions of actuation engagement structures of the instrument input adapter, and the positions of elements therein, corresponding to particular phases of engagement of the instrument input adapter with and disengagement of the instrument input adapter from the instrument output adapter in accordance with an embodiment of the present disclosure.

FIG. 13A illustrates an alternate embodiment of a docking station and a corresponding translation unit in accordance with the present disclosure.

FIG. 13B illustrates yet another embodiment of a docking station and a corresponding translation unit in accordance with an embodiment of the present disclosure.

FIG. 13C provides a cross-sectional front view through portions of a docking station configured for carrying a set of cradle or drum structures that are rotatably coupled to actuators by which roll motion is individually providable to one or more actuation assemblies and/or an imaging endoscope.

FIG. 14A illustrates a representative single actuator/motor per DOF configuration, and potential backlash-like effects that can be associated therewith.

FIG. 14B illustrates a representative dual actuator/motor per DOF configuration in accordance with an embodiment of the present disclosure, and a reduction or minimization of backlash-like effects as a result of such a configuration.

FIG. 15 is an illustration of an offline/online fixed tensioning technique, procedure, or process in accordance with an embodiment of the present disclosure.

FIG. 16A is an illustration of an active pretensioning technique, procedure, or process in accordance with an embodiment of the present disclosure, and FIG. 16B is a representative graph of actuator/motor position and torque corresponding thereto.

FIGS. 16C-16F are graphs respectively indicating measured motor position, measured motor velocity, measured motor torque, and the first derivative of measured motor torque for a first actuator/motor of a particular actuator/motor pair with respect to time during while performing the active pretensioning technique of FIG. 16A.

FIG. 16G-16J are graphs respectively indicating measured motor position, measured motor velocity, measured motor torque, and the first derivative of measured motor torque for a second actuator/motor of the actuator/motor pair under consideration with respect to time during while performing the active pretensioning technique of FIG. 16A.

FIGS. 17 and 18 are schematic illustrations showing portions of a crimp-free pulley-based roll joint primitive in accordance with an embodiment of the present disclosure.

### DETAILED DESCRIPTION

In the present disclosure, depiction of a given element or consideration or use of a particular element number in a particular FIG. or a reference thereto in corresponding descriptive material can encompass the same, an equivalent, or an analogous element or element number identified in another FIG. or descriptive material associated therewith. The use of “/” in a FIG. or associated text is understood to mean “and/or” unless otherwise indicated. The recitation of a particular numerical value or value range herein is understood to include or be a recitation of an approximate numerical value or value range, for instance, within +/−20%, +/−15%, +/−10%, or +/−5%.

As used herein, the term “set” corresponds to or is defined as a non-empty finite organization of elements that mathematically exhibits a cardinality of at least 1 (i.e., a set as defined herein can correspond to a unit, singlet, or single element set, or a Multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). In general, an element of a set can include or be a system, an apparatus, a device, a structure, an object, a process, a physical parameter, or a value depending upon the type of set under consideration.

Embodiments of the present disclosure are directed to master-slave flexible robotic endoscopy systems, which include a master-side system and a slave-side system that is controllable or controlled by the master-side system. Depending upon embodiment details, one or more portions of a master-slave flexible robotic endoscopy system in accordance with the present disclosure can correspond or be analogous to or include one or more types of elements, structures, and/or devices described (a) in International Patent Application No. PCT/SG2013/000408; and/or (b) International Patent Publication No. WO 2010/138083.

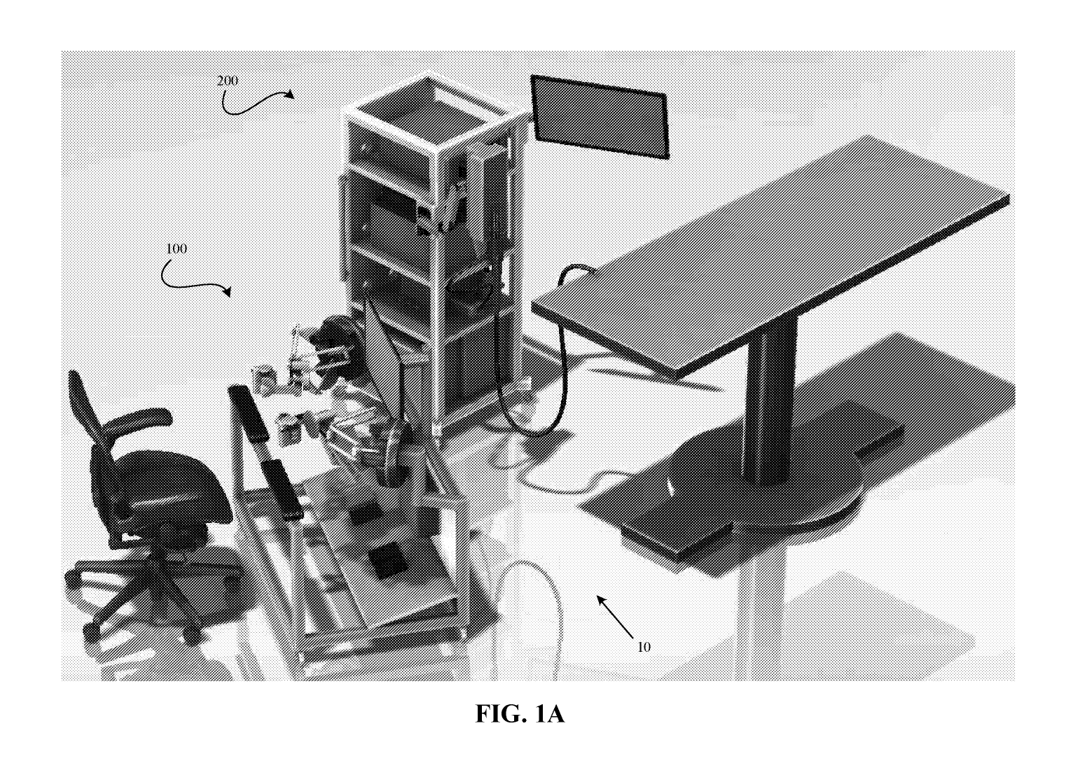

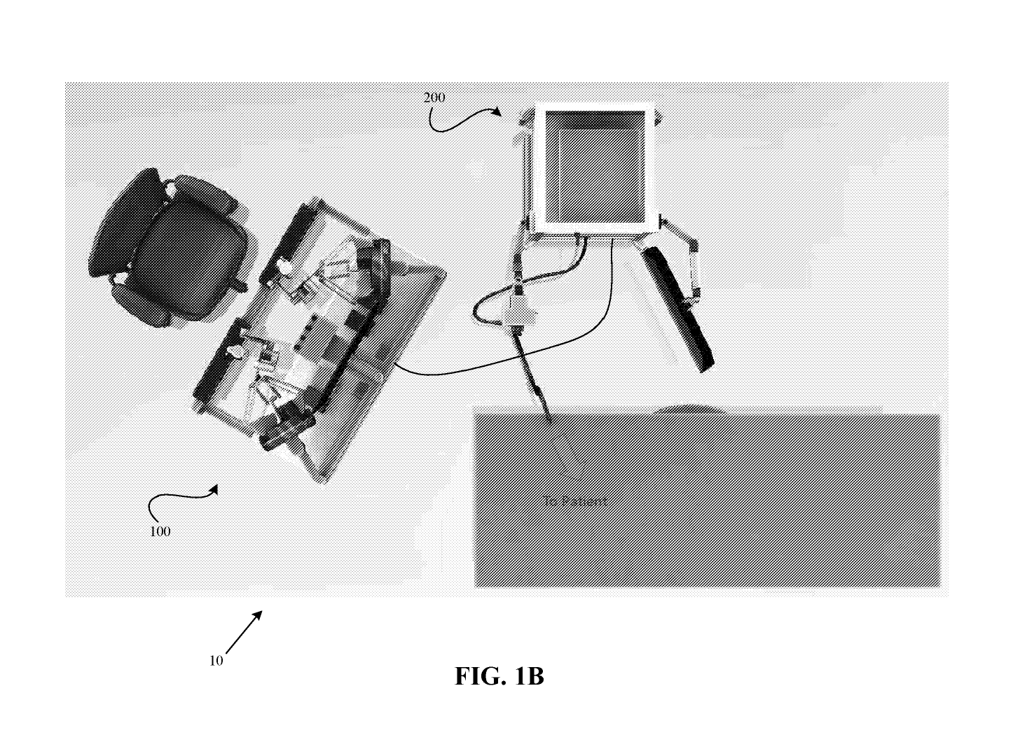

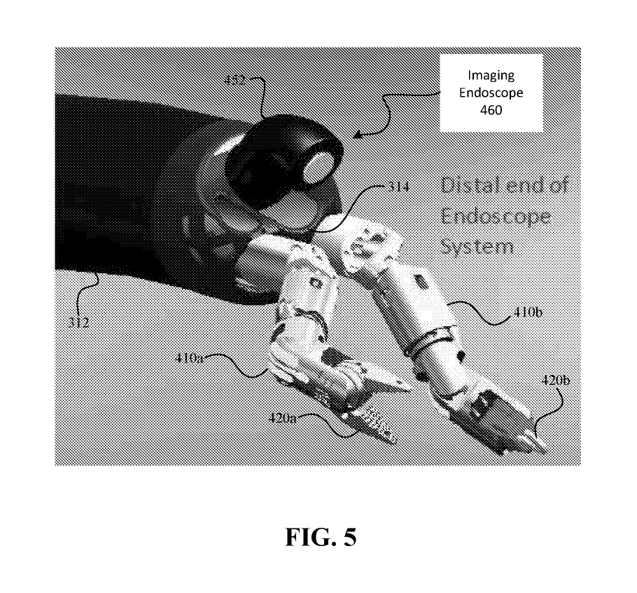

FIGS. 1A and 1B are schematic illustrations of a master-slave flexible robotic endoscopy system 10 in accordance with an embodiment of the disclosure. In an embodiment, the system 10 includes a master or master-side system 100 having master-side elements associated therewith, and a slave or slave-side system 200 having slave-side elements associated therewith. With further reference to FIG. 5, in various embodiments, the master system 100 and the slave system 200 are configured for signal communication with each other such that the master system 100 can issue commands to the slave system 200 and the slave system 200 can precisely control, maneuver, manipulate, position, and/or operate (a) a set of robotic arms 400a,b and corresponding end effectors 410a,b carried or supported by an endoscope 300 (also referred to herein as a transport endoscope 300) of the slave system 200, and possibly (b) an imaging endoscope or imaging probe member 460 carried or supported by the transport endoscope 300, in response to master system inputs.

In various embodiments, the imaging endoscope or imaging probe member 460 is typically configured for at least surge displacement and possibly also roll motion (e.g., about a central or longitudinal axis of the imaging endoscope or imaging probe member 460) in response to control signals received from the master system 100 and/or a set of control's carried by the transport endoscope 300. In some embodiments, the imaging endoscope/imaging probe member 460 is configured for heave, sway, and/or pitch motion, such as by way of internally carried tendons, in which case the imaging endoscope/imaging probe member 460 can be referred to as a robotically controlled imaging endoscope/imaging probe member 460. Control signals for spatially manipulating a robotically controlled imaging endoscope 460/imaging probe member 460 can be generated by the master system 100, and/or a set of slave system controls, such as control buttons, switches, a joystick, or the like carried by the transport endoscope 300.

The master and slave systems 100, 200 can further be configured such that the slave system 200 can dynamically provide tactile/haptic feedback signals (e.g., force feedback signals) to the master system 100 as the robotic arms 410a,b and/or end effectors 420a-b associated therewith are positioned, manipulated, or operated. Such tactile/haptic feedback signals are correlated with or correspond to forces exerted upon the robotic arms 410a,b and/or end effectors 420a-b within an environment in which the robotic arms 410a,b and end effectors 420a,b reside.

Various embodiments in accordance with the present disclosure are directed to surgical situations or environments, for instance, Natural Orifice Transluminal Endoscopic Surgery (NOTES) procedures performed upon a patient or subject while they are disposed on a surgical table or platform 20. In such embodiments, at least portions of the slave system 200 are configured to reside within an Operating Theatre (OT) or Operating Room (OR). Depending upon embodiment details, the master system 100 can reside within or outside of (e.g., near or remote from) the OT/OR. Communication between the master system 100 and the slave system 200 can occur directly (e.g., through a set of local communication lines, and/or local wireless communication), or indirectly by way of one or more networks (e.g., a Local Area Network (LAN), a Wide Area Network (WAN), and/or the Internet) in accordance with embodiment details.

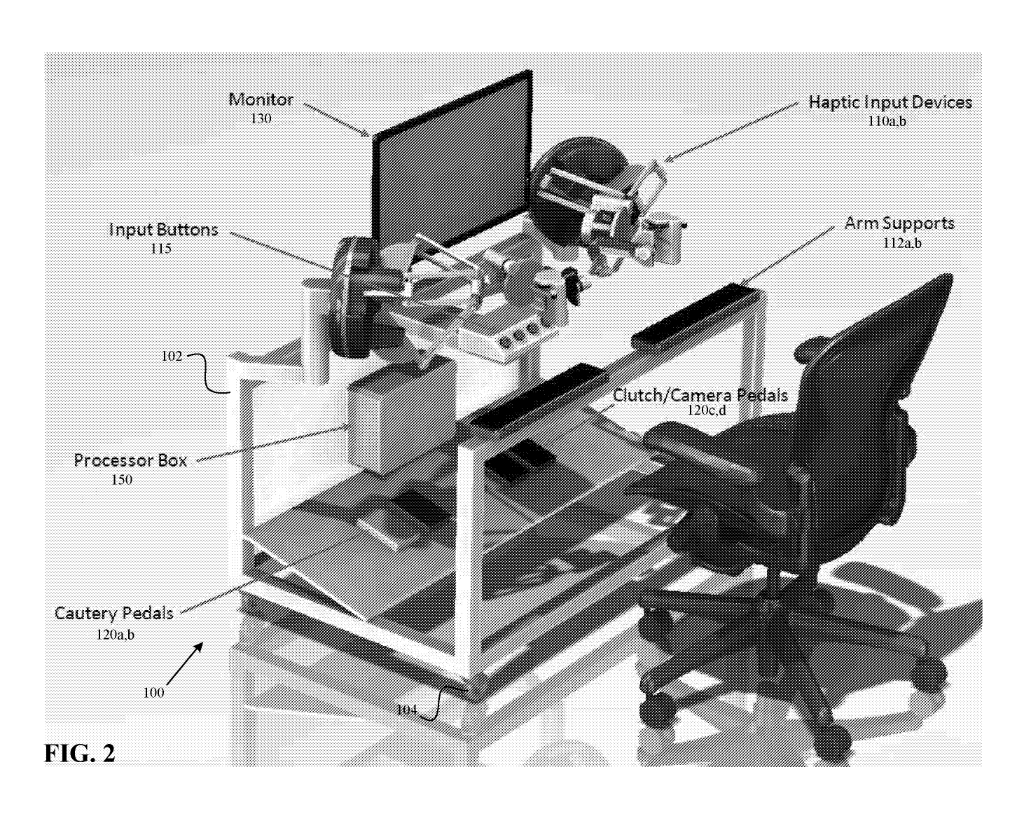

FIG. 2 is a schematic illustration of a master system 100 in accordance with an embodiment of the present disclosure. In an embodiment, the master system 100 includes a frame or console structure 102 that carries left and right haptic input devices 110a,b; a set of additional/auxiliary hand-operated input devices/buttons 115; a set of foot operated controls or pedals 120a-d; a display device 130; and a processing module 150. The frame/console structure 102 can include a set of wheels 104 such that the master system 100 is readily portable/positionable within an intended usage environment (e.g., an OT/OR, or a room external to or remote therefrom); and a set of arm supports 112. During a representative endoscopy procedure, a surgeon positions or seats themselves relative to the master system 100 such that their left and right hands can hold or interact with the left and right haptic input devices 110a,b, and their feet can interact with the pedals 120a-d. The processing module 150 processes signals receive from the haptic input devices 110a,b, the additional/auxiliary hand-operated input devices 115, and the pedals 120a-d, and issues corresponding commands to the slave system 200 for purpose of manipulating/positioning/controlling the robotic arms 410a,b and the end effectors 420a,b corresponding thereto, and possibly manipulating/positioning/controlling the imaging endoscope 460. The processing module 150 can additionally receive tactile/haptic feedback signals from the slave system 200, and conveys such tactile/haptic feedback signals to the haptic input devices 110a,b. The processing module 150 includes computing/processing and communication resources (e.g., one or more processing units, memory/data storage resources including Random Access Memory (RAM) Read-only Memory (ROM), and possibly one or more types of disk drives, and a serial communication unit and/or network communication unit) in a manner readily understood by one having ordinary skill in the relevant art.

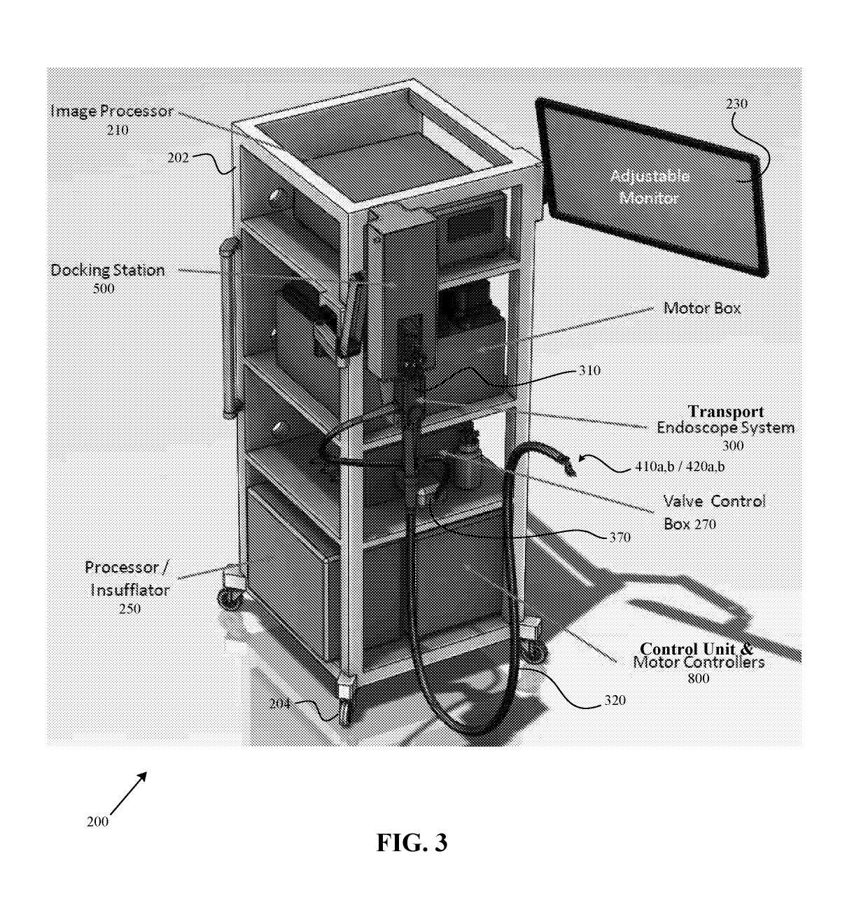

FIG. 3 is a schematic illustration of a slave system 200 in accordance with an embodiment of the present disclosure. In an embodiment, the slave system 200 includes an endoscope or transport endoscope 300 having a flexible elongate shaft 312; a docking station 500 to which the transport endoscope 300 can be selectively/selectably coupled (e.g., mounted/docked and dismounted/undocked); an imaging subsystem 210; an endoscopy support function subsystem 250 and an associated valve control unit 270; an actuation unit or motorbox 600; and a main control unit 800. In several embodiments, the slave system 200 additionally includes a patient-side cart, stand, or rack 202 configured for carrying at least some slave system elements. The patient side cart 202 typically includes wheels 204 to facilitate easy portability and positioning of the slave system 200 (e.g., at a desired location within an OT/OR).

In brief, the imaging subsystem 210 facilitates the provision or delivery of illumination to the imaging endoscope 460, as well as the processing and presentation of optical signals captured by the imaging endoscope 460. The imaging subsystem 210 includes an adjustable display device 220 configured for presenting (e.g., on a real-time basis) images captured by way of the imaging endoscope 460, in a manner readily understood by one having ordinary skill in the relevant art. The endoscopy support function subsystem 250 in association with the valve control unit 270 facilitates the selective controlled provision of insufflation or positive pressure, suction or negative/vacuum pressure, and irrigation to the transport endoscope 300, as also readily understood by one having ordinary skill in the relevant art. The actuation unit/motorbox 600 provides a plurality of actuators or motors configured for driving the robotic arms 410a,b and the end effectors 420a,b under control of the main control unit 800, which includes a set of motor controllers.

The main control unit 800 additionally manages communication between the master system 100 and the slave system 200, and processes input signals received from the master system 100 for purpose of operating the robotic arms 410a,b and end effectors 420a,b in a manner that directly and precisely corresponds to surgeon manipulation of the master system's haptic input devices 110a,b. In multiple embodiments, the main control unit 800 additionally generates the aforementioned tactile/haptic feedback signals, and communicates such tactile/haptic feedback signals to the master system 100 on a real-time basis. In several embodiments, the tactile/haptic feedback signals can be generated by way of sensors that are disposed proximal to the transport endoscope's shaft 312 and/or body 310 (e.g., sensors that reside in the motorbox 600), without use or exclusive of sensors carried within or distal to the transport endoscope's shaft 312 and/or body 310 (e.g., sensors carried on, near, or generally near a robotic arm 410 or end effector 420). The main control unit 800 includes signal/data processing, memory/data storage, and signal communication resources (e.g., one or more microprocessors, RAM, ROM, possibly a solid state or other type of disk drive, and a serial communication unit and/or network interface unit) in a manner readily understood by one having ordinary skill in the relevant art.

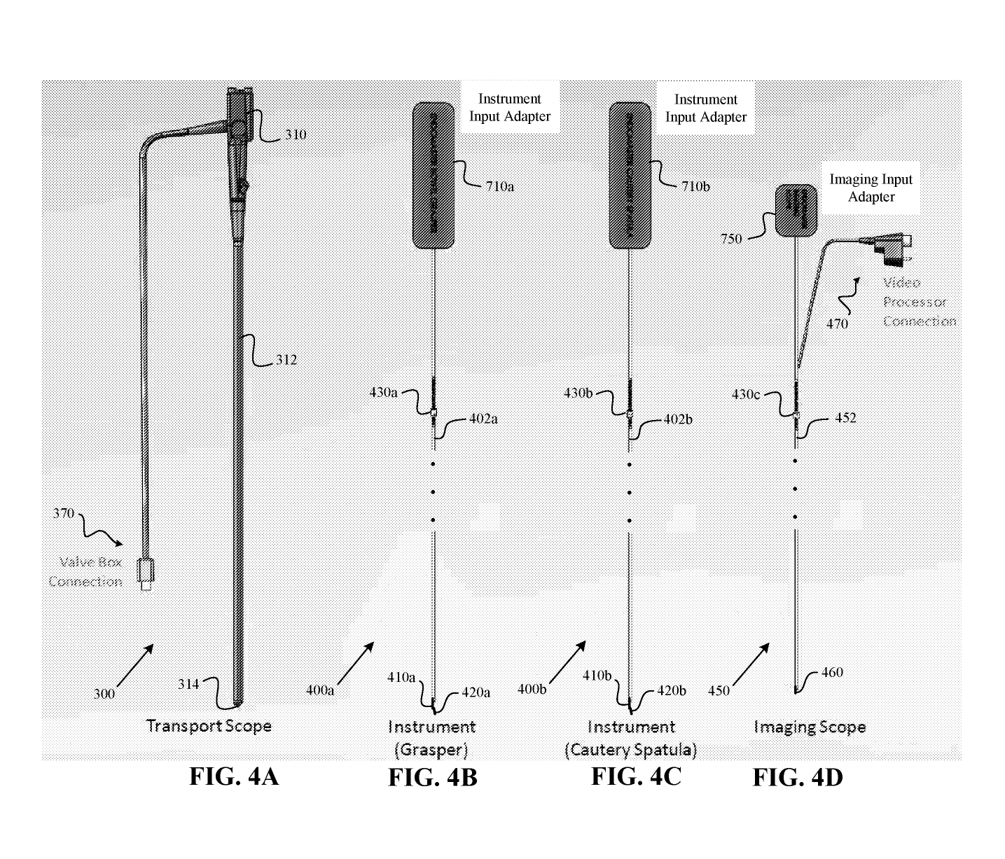

With further reference to FIGS. 4A-4D, the transport endoscope 300 includes a main body or housing 310 from which the flexible elongate shaft 312 extends. The transport endoscope 300 additionally includes an endoscopy support function connector assembly 370 by which the transport endoscope's main body 310 can be coupled to the endoscopy support function subsystem 250, in a manner readily understood by one having ordinary skill in the relevant art.

The main body 310 defines a proximal portion, border, surface, or end of the transport endoscope 300, and provides a number of apertures, openings, or ports through which channels or passages that extend within and along the transport endoscope's shaft 312 are accessible. In several embodiments, the main body 310 additionally provides a control interface for the transport endoscope 300, by which an endoscopist can exert navigational control over the transport endoscope's shaft 312. For instance, the main body 310 can include a number of control elements, such as one or more buttons, knobs, switches, levers, joysticks, and/or other control elements to facilitate endoscopist control over transport endoscope operations, in a manner readily understood by one having ordinary skill in the relevant art.

The shaft 312 terminates at a distal end 314 of the transport endoscope 300, and the channels/passages within the shaft 312 terminate at openings or apertures disposed at, proximate, or near the shaft's distal end 314. In various embodiments, the channels/passages provided by the transport endoscope 300 include a set of instrument channels, plus passages for enabling the delivery of insufflation or positive pressure, suction or vacuum pressure, and irrigation to an environment in which the distal end of the shaft 312 resides.

The set of instrument channels includes at least one channel configured for carrying portions of a flexible actuation assembly 400 that can be inserted into and withdrawn from the transport endoscope 300. Each actuation assembly 400 includes a robot arm 410 and an end effector 420 corresponding thereto; flexible control elements, tendon elements, or tendons by which the robot arm 410 and the end effector 420 can be positioned or manipulated in accordance with a predetermined number of DOF; and an interface or adapter by which the actuation assembly's flexible tendons can be mechanically coupled to and decoupled from specific actuators within the motorbox 600. In various embodiments, each tendon resides within a corresponding flexible sheath (e.g., a helical coil). A given tendon and its corresponding sheath can be defined as a tendon/sheath element. In a number of embodiments, an actuation assembly 400 can be disposable.

In an embodiment indicated in FIGS. 4A-4B, a given actuation assembly 400a,b includes a robot arm 410a,b and its corresponding end effector 420a,b; a flexible elongate outer sleeve and/or coil 402a,b that internally carries a plurality of tendon/sheath elements, such that tension or mechanical forces can be selectively applied to particular tendon elements for precisely manipulating and controlling the operation of the robot arm 410a,b and/or the end effector 420a,b; and an instrument input adapter 710a,b by which tendons within the outer sleeve 402a,b can be mechanically coupled to corresponding actuators within the motorbox 600, as further detailed below.

The robot arm 410a,b, end effector 420a,b, and portions of the outer sleeve/coil 402a,b can be inserted into an instrument channel of the transport endoscope's shaft 312, such that the robot arm 410a,b and the end effector 420a,b reach or approximately reach, and can extend a predetermined distance beyond, the distal end 314 of the shaft 312. As described in detail below, the actuation assembly's outer sleeve/coil 402a,b, and hence the robot arm 410a,b and end effector 420a,b, can be selectively longitudinally translated or surged (i.e., displaced distally or proximally with respect to the distal end 314 of the transport endoscope's shaft 312) by way of a translation module, unit, stage, or mechanism such that the proximal-distal positions of the robotic arm 410a,b and the end effector 420a,b relative to the distal end 314 of the shaft 312 can be adjusted within an environment beyond the distal end 314 of the shaft 312, up to a predetermined maximum distance away from the distal end 314 of the shaft 312, for purpose of carrying out an endoscopic procedure.

In particular embodiments, the actuation assembly 400a,b includes a collar element, collet, or band 430a,b that surrounds at least a portion of the outer sleeve/coil 402a,b at a predetermined distance away from the distal tip of the end effector 420a,b. As detailed below, the collar element 430a,b is designed to matingly engage with a receiver of the translation mechanism, such that longitudinal/surge translation of the collar element 430a,b across a given distance relative to the distal end of the shaft 312 results in corresponding longitudinal/surge translation of the robotic arm 410a,b and end effector 420a,b.

In several embodiments, the channels/passages provided within the transport endoscope's shaft 312 additionally include an imaging endoscope channel, which is configured for carrying portions of a flexible imaging endoscope assembly 450 that can be inserted into and withdrawn from the transport endoscope 300, where the flexible imaging endoscope assembly 450 corresponds to or includes at least portions of an imaging endoscope/imaging probe member 460. In a manner analogous or generally analogous to that described above for the actuation assembly 400a,b, in an embodiment the imaging endoscope assembly 450 includes a flexible outer sleeve, coil, or shaft 452 that surrounds or forms an outer surface of the flexible imaging endoscope 460; possibly an imaging input adapter 750 by which a set of tendons corresponding to or within the imaging endoscope 460 can be mechanically coupled to corresponding actuators within the motorbox 600 such that a distal portion of the imaging endoscope 460 can be selectively maneuvered or positioned in accordance with one or more DOFs (e.g., heave and/or sway motion) within an environment at, near, and/or beyond the distal end 314 of the transport endoscope's shaft 312; and an imaging connector assembly 470 by which electronic and/or optical elements (e.g., optical fibers) of the imaging endoscope 460 can be respectively electronically and/or optically coupled to an image processing unit of the imaging subsystem 210. For instance, in some embodiments the imaging endoscope 460 can include or be coupled to tendons such that a distal end or face of the imaging endoscope 460 can selectively/selectably capture anterograde and retrograde images of the robotic arms 410a,b and end effectors 420a,b during an endoscopic procedure. In some embodiments, the imaging endoscope assembly 450 can be disposable.

In a manner identical, essentially identical, or analogous to that for the actuation assembly 400a,b, the outer sleeve 452 of the imaging endoscope assembly 450, and hence the distal end of the imaging endoscope 460, can be selectively longitudinally translated/surged relative to the distal end 314 of the transport endoscope's shaft 312 by way of the translation mechanism, such that the longitudinal or proximal-distal position of the imaging endoscope 460 can be adjusted at, near, and/or beyond the distal end of the shaft 312 across a predetermined proximal-distal distance range in association with an endoscopic procedure. In a number of embodiments, the imaging endoscope assembly 400 includes a collar element 430c that surrounds at least portions of the imaging endoscope assembly's outer sleeve 452 at a predetermined distance away from the distal end of the imaging endoscope 450. The collar element 430c is configured for mating engagement with a receiver of the translation mechanism, such that longitudinal/surge displacement of the collar element 430c across a given distance relative to the distal end of the transport endoscope's shaft 312 results in corresponding longitudinal/surge displacement of the distal end of the imaging endoscope 460.

As indicated above, the actuation assemblies 400a,b and the imaging endoscope assembly 450 are configured for insertion into and withdrawal from instrument channels and an imaging endoscope channel of the transport endoscope 300, respectively. When the actuation assemblies 400a,b and the imaging endoscope assembly 450 have been fully inserted into the transport endoscope 300 prior to their manipulation in an environment external to the distal end 314 of the transport endoscope shaft 312 during an endoscopic procedure, each collar element 430a-c remains outside of and at least slightly away from the transport endoscope's shaft 312, and in various embodiments outside of and at least slightly away from the transport endoscope's main body 310, such that longitudinal translation or surge motion of a given collar element 430a-c across a predetermined proximal-distal distance range can freely occur by way of the translation unit, without interference from the transport endoscope's shaft 312 and/or main body 310.

Thus, the outer sleeve/coil 402a,b of each actuation assembly 400a,b must distally extend a sufficient length away from a distal border of its collar element 430a,b, such that the end effector 420a,b reaches or approximately reaches the distal end 314 of the transport endoscope's shaft 312 when the collar element 430a,b resides at a most-proximal position relative to the translation unit. Similarly, the imaging endoscope assembly's outer sleeve 452 must distally extend a sufficient length away from its collar element 430c such that the distal end of the imaging endoscope 460 resides at an intended position at, proximate to, or near the distal end 314 of the transport endoscope's shaft 312 when the collar element 430c is at a most-proximal position relative to the translation unit.

In a number of embodiments, the transport endoscope 300 is configured for carrying two actuation assemblies 400a,b, plus a single imaging endoscope assembly 450. Each actuation assembly 400a,b typically corresponds to a given type of endoscopic tool. For instance, in a representative implementation, a first actuation assembly 400a can carry a first robotic arm 410a having a grasper or similar type of end effector 420a; and a second actuation assembly 400b can carry a second robotic arm 410b having a cautery spatula or similar type of cauterizing end effector 420b.

In certain embodiments, the transport endoscope 300 can be configured for carrying another number of actuation assemblies 400. Furthermore, the cross-sectional dimensions of the transport endoscope 300, the channels/passages therein, one or more actuation assemblies 400, and/or an imaging endoscope assembly 450 can be determined, selected, or specified in accordance with a given type of surgical/endoscopic procedure and/or transport endoscope shaft size/dimensional constraints under consideration.

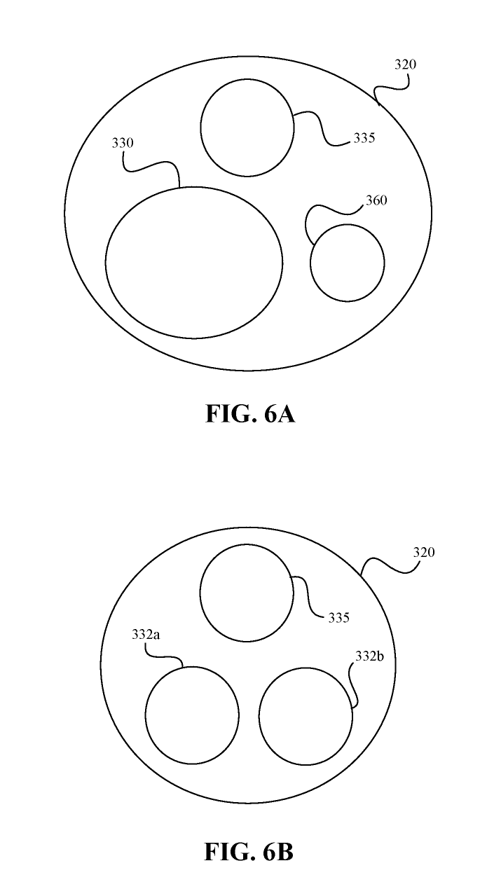

FIG. 6A is a representative cross sectional illustration of a transport endoscope shaft 312 in accordance with another embodiment of the present disclosure, in which the channels/passages therein include a primary instrument channel 330 having a large or maximal cross-sectional area/diameter configured for accommodating a high/maximum DOF robot arm/end effector 410, 420; a secondary instrument channel 360 having a smaller or significantly smaller cross-sectional area/diameter than the primary instrument channel 330, which can be configured for accommodating a manually operated conventional endoscopic instrument/tool, such as a conventional grasper (e.g., in such an embodiment, a robotic actuation assembly 400 as well as a conventional/manual actuation assembly can be inserted into corresponding ports in the transport endoscope body 310); and an imaging endoscope channel 335 configured for accommodating an imaging endoscope 460.

FIG. 6B is a representative cross sectional illustration of a transport endoscope shaft 312 in accordance with yet another embodiment of the present disclosure, in which the channels/passages therein include a first and a second instrument channel 332a,b having relatively small(er) cross-sectional areas or diameters configured for accommodating reduced/limited DOF robotic arms/end effectors 410a,b, 420a,b compared to the transport endoscope shaft embodiment of FIG. 6A; and an imaging endoscope channel 335 configured for accommodating an imaging endoscope 460.

Transport endoscope shaft embodiments such as those shown in FIGS. 6A and 6B can result in smaller overall cross-sectional areas than a transport endoscope shaft 312 described elsewhere herein, for purpose of facilitating an given type of endoscopic procedure and/or improving intubation, in a manner readily understood by one having ordinary skill in the relevant art.

### Representative Procedural Setup and Interface Coupling to Motorbox

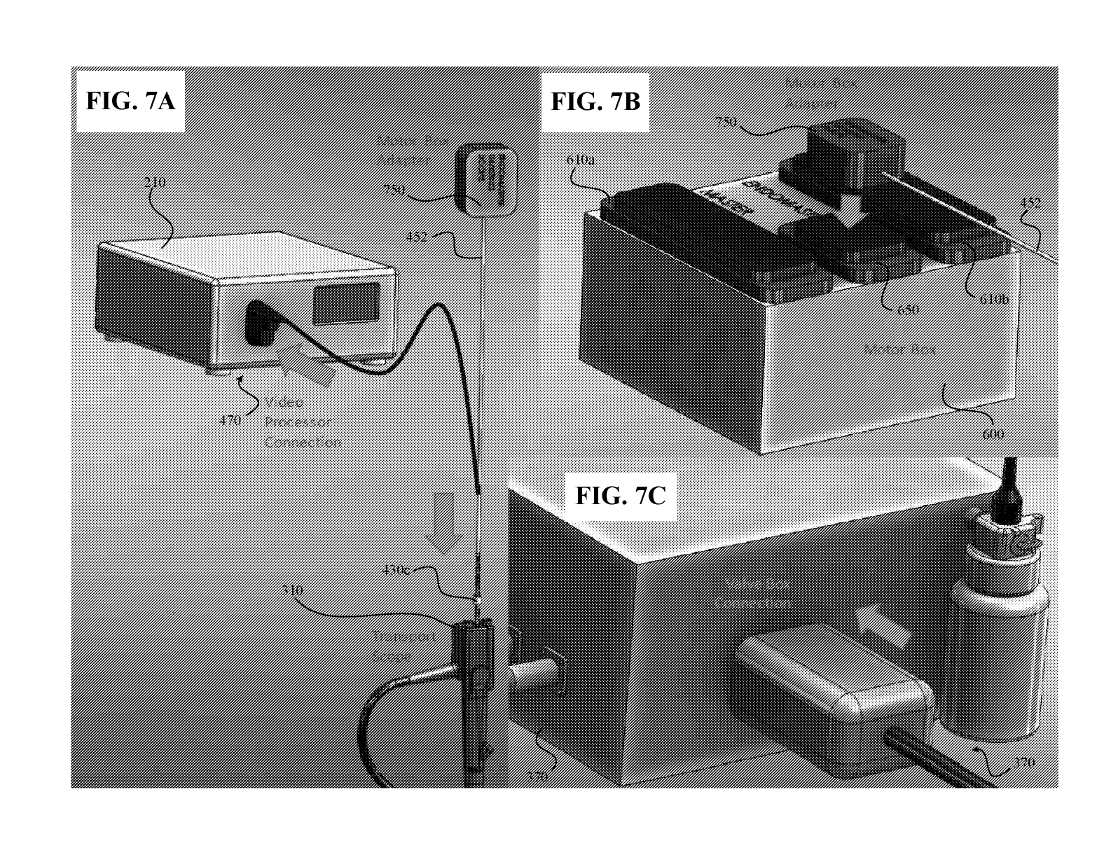

FIGS. 7A-9 illustrate portions of a representative setup procedure by which an imaging endoscope assembly 450 and a pair of actuation assemblies 400a,b can be inserted into the transport endoscope 300 and coupled to or interfaced with other portions of the slave system 200, including the motorbox 600.

As indicated in FIG. 7A, portions of the imaging endoscope assembly's outer sleeve 452 distal to the collar element 430c corresponding thereto can be inserted into an intended or appropriately dimensioned aperture or port formed in the transport endoscope's body 310, such that the imaging endoscope 460 can be fed into and distally advanced along the transport endoscope's shaft 312 to an initial intended, default, or parked position relative to the distal end 314 thereof. As previously indicated, the collar element 430c coupled to the imaging endoscope assembly's outer sleeve 452 remains external to the transport endoscope's shaft 312. More particularly, in the embodiment shown, the collar element 430c remains external to the transport endoscope's body 310, such that the collar element 430c resides a given distance proximate to the port that received the outer sleeve 452 of the imaging endoscope assembly 450. The imaging connector assembly 470 can be coupled to the imaging subsystem 210, for instance, as in a manner indicated in FIG. 7A, as readily understood by one having ordinary skill in the relevant art, such that the imaging endoscope 460 can output illumination and capture images.

As further indicated in FIG. 7B, the imaging endoscope assembly's imaging input adapter 750 can be coupled to a corresponding imaging output adapter 650 of the motorbox 600. By way of such adapter-to-adapter coupling, a set of tendons internal to the imaging endoscope assembly's outer sleeve 452 can be mechanically coupled or linked to one or more actuators or motors within the motorbox 600. Such tendons are configured for positioning or maneuvering the imaging endoscope 460 in accordance with one or more DOFs. Consequently, the imaging endoscope 460 can be selectively positioned or manipulated in particular manners relative to the distal end 314 of the transport endoscope's shaft 312 as a result of the selective application of tension to the imaging endoscope assembly's tendons by way of one or more actuators within the motorbox 600 that are associated with imaging endoscope position control.

In addition to the foregoing, the transport endoscope's support function connector assembly 370 can be coupled to the endoscopy support function subsystem 270, for instance, in a manner indicated in FIG. 7C, in order to facilitate the provision of insufflation or positive pressure, suction or negative/vacuum pressure, and irrigation in a manner readily understood by an individual having ordinary skill in the relevant art.

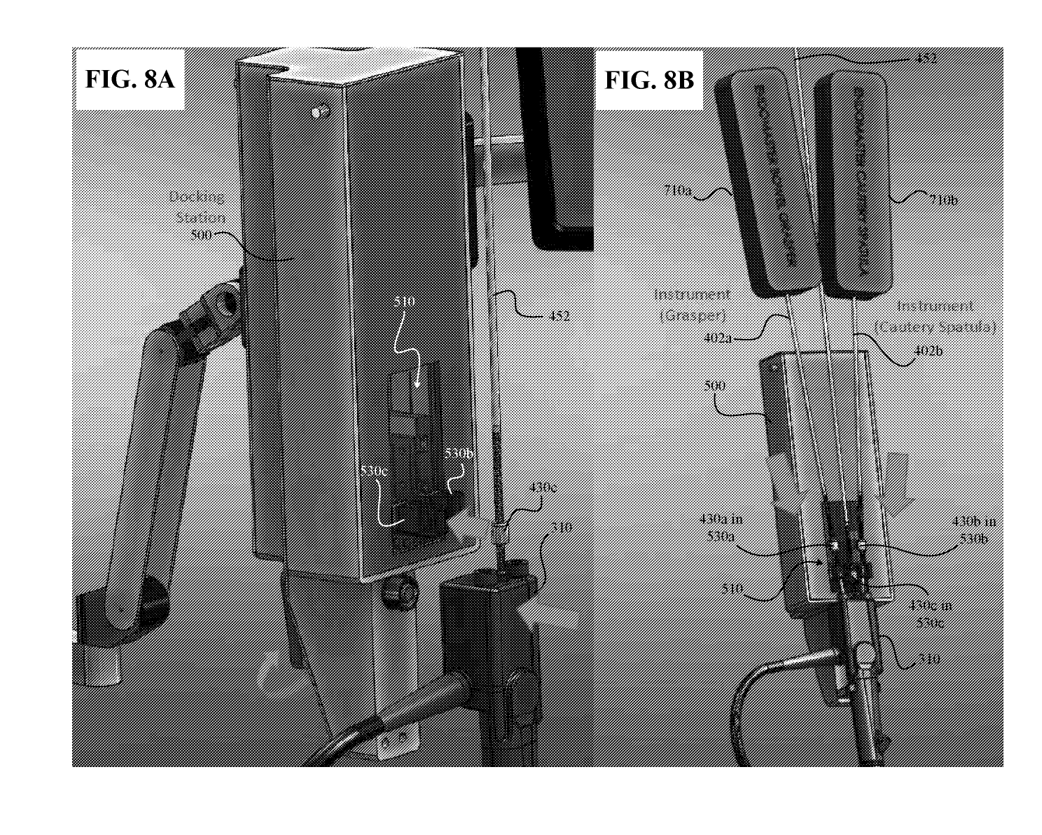

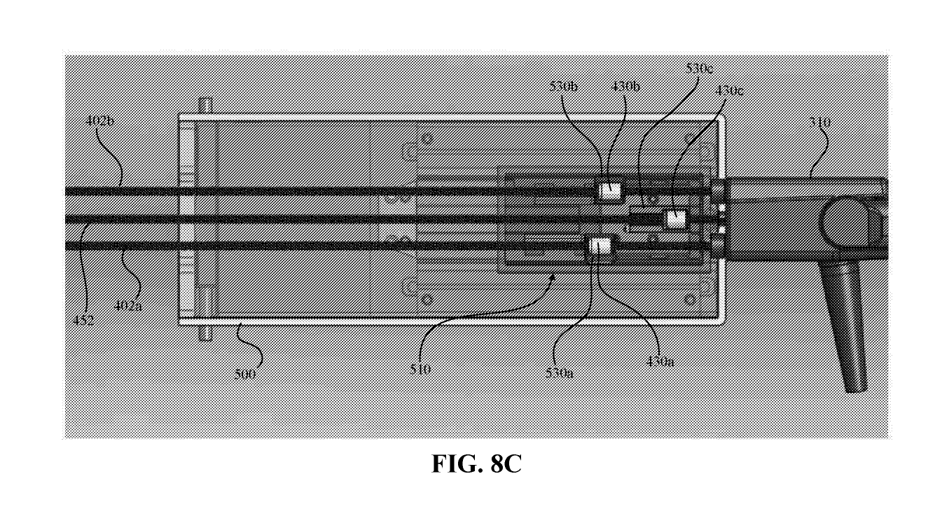

With reference to FIG. 8A, the transport endoscope's body 310 can be docked or mounted to the docking station 500, and the imaging endoscope assembly's collar element 430c can be inserted into or matingly engaged with a corresponding receiver or clip 530c provided by a translation unit 510 associated with the docking station 500. Once the imaging endoscope assembly's collar element 430c is securely held by its corresponding clip 530c, the imaging endoscope assembly's sleeve 452 can be selectively/selectably longitudinally translated or surged by the translation unit 510 across a predetermined proximal-distal distance range, as further detailed below, for instance, in response to surgeon manipulation of a haptic input device 110a,b or other control (e.g., a foot pedal) at the master station 100, and/or endoscopist manipulation of a control element on the transport endoscope's body 310 (e.g., where surgeon input can override endoscopist input directed to longitudinally translating/surging the imaging endoscope 460).

With further reference to FIG. 8B, in a manner analogous to that described above, portions of each actuation assembly 400a,b distal to a corresponding actuation assembly collar element 430a,b can be inserted into an intended/appropriately dimensioned port within the body 310 of the transport endoscope 300. As a result, each robot arm 410a,b and end effector 420a,b can be fed into and distally advanced along the transport endoscope's shaft 312 toward and to an initial intended, default, or parked position relative to the shaft's distal end 314. The collar element 430a,b carried by each actuation assembly's outer sleeve/coil 402a,b remains external to the transport endoscope's shaft 312, and in several embodiments external to the transport endoscope's body 310, such that each collar element 430a,b resides a given distance proximate to the port that received the outer sleeve/coil 402a,b of the actuation assembly 400a,b.

In a manner analogous to that for the imaging endoscope assembly 450, each actuation assembly's collar element 430a,b can be inserted into or matingly engaged with a corresponding receiver or clip 530a,b provided by the translation unit 510. Once each such collar element 430a,b is securely retained by its corresponding clip 530a,b, the translation unit 510 can selectively/selectably longitudinally translate or surge one or both of the actuation assemblies 400a,b (e.g., in an independent manner) across a predetermined proximal-distal distance range, for instance, in response to surgeon manipulation of one or both haptic input devices 110a,b at the master station 100.

FIG. 8C is a schematic illustration showing a representative translation unit 510 associated with or carried by the docking station 500, and a representative manner in which the collar elements 430a-c corresponding to the actuation assemblies 400a,b and the imaging endoscope assembly 450 are retained by corresponding translation unit clips 530a-c. The translation unit 510 can include an independently adjustable/displaceable translation stage corresponding to each actuation assembly 400a,b as well as the imaging endoscope assembly 450. In a representative implementation, a given translation stage can include or be a ball screw or a linear actuator configured for providing longitudinal/surge displacement to a corresponding clip 530 across a predetermined maximum distance range, in a manner readily understood by one having ordinary skill in the relevant art.

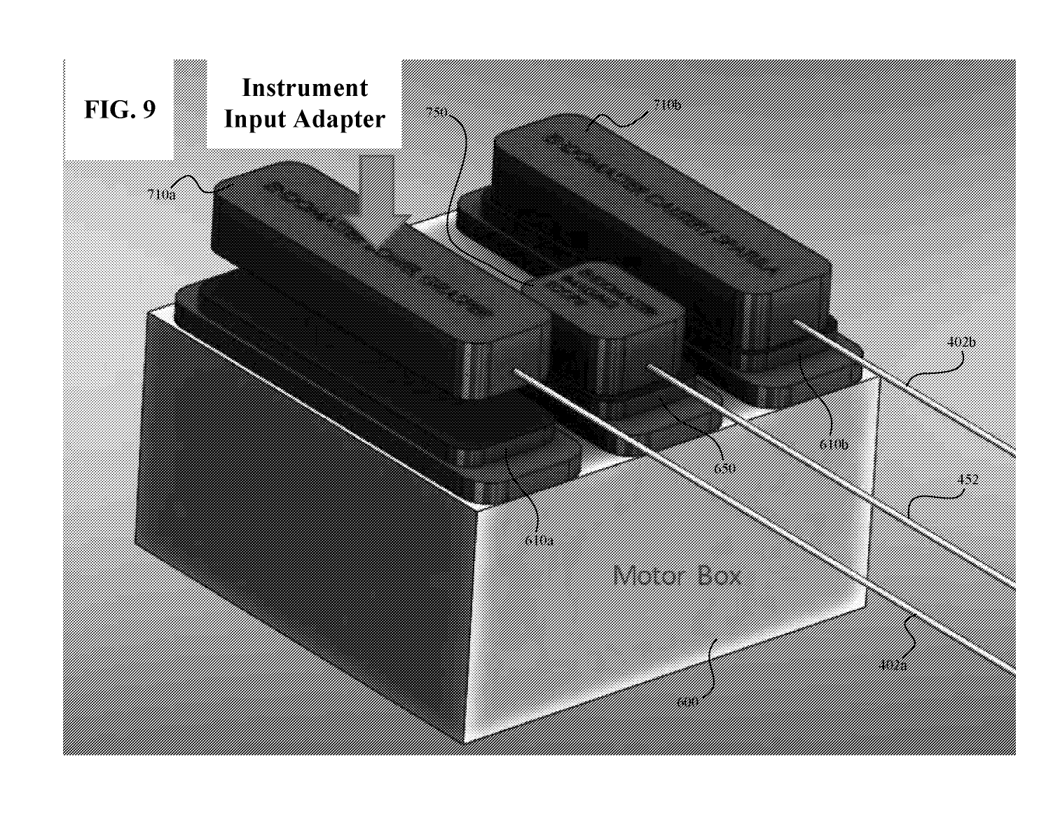

FIG. 9 is a schematic illustration showing coupling of each actuation assembly's instrument input adapter 710a,b to a corresponding instrument output adapter 610a,b of the motorbox 600 in accordance with an embodiment of the present disclosure. By way of such adapter-to-adapter coupling, tendons internal to each actuation assembly's outer sleeve/coil 402a,b can be mechanically coupled or linked to particular actuators or motors within the motorbox 600. For any given actuation assembly 400, such tendons are configured for positioning or maneuvering the robot arm 410a,b and corresponding end effector 420a,b in accordance with predetermined DOFs. Consequently, each actuation assembly's robot arm 410a,b and end effector 402a,b can be selectively positioned or manipulated relative to the distal end 314 of the transport endoscope's shaft 312 as a result of the selective application of tension to the tendons within the actuation assembly 400a,b by way of one or more actuators/motors within the motorbox 600 that are associated with robot arm/end effector position control. Moreover, such adapter-to-adapter coupling enables the establishment, re-establishment, or verification of intended, desired, or predetermined tension levels in the tendons within each actuation assembly 400a,b prior to the initiation of an endoscopic procedure (e.g., tendon pretension levels), and in some embodiments on-the-fly establishment or adjustment of tendon tension levels during an endoscopic procedure. Furthermore, in various embodiments, such adapter-to-adapter coupling enables the maintenance of a given or predetermined tension level (e.g., a predetermined minimum tension level) in actuator assembly tendons when the instrument input adapter 710a,b is not engaged with, or disengaged from, the instrument output adapter 610a,b, as further detailed hereafter.

### Representative Input Adapter and Output Adapter Structures and Couplings

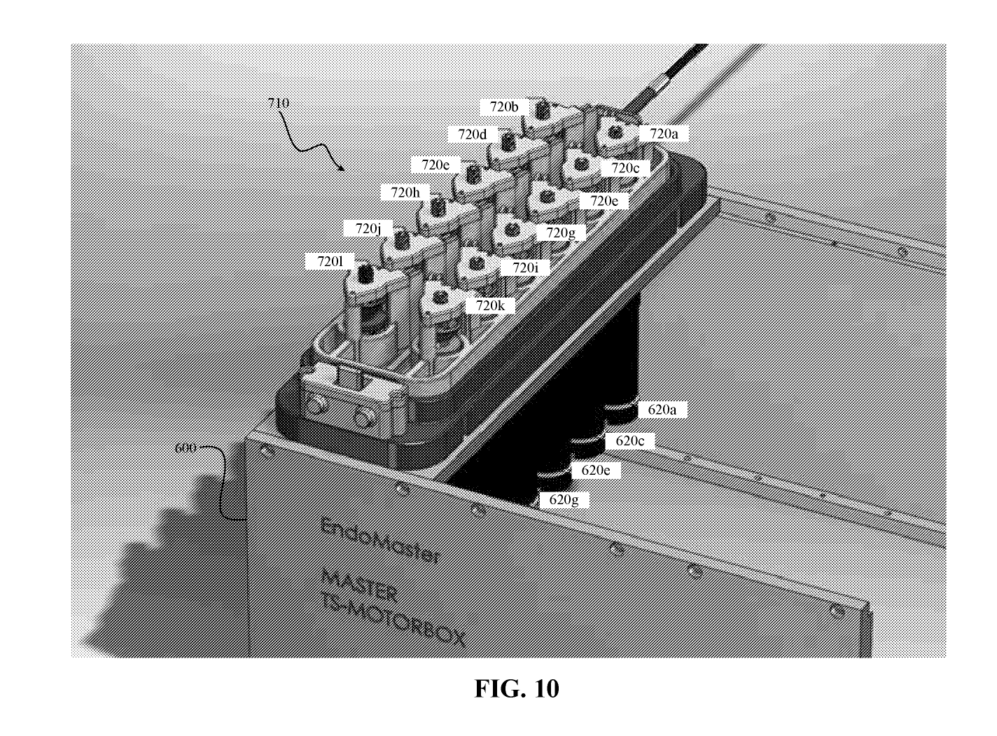

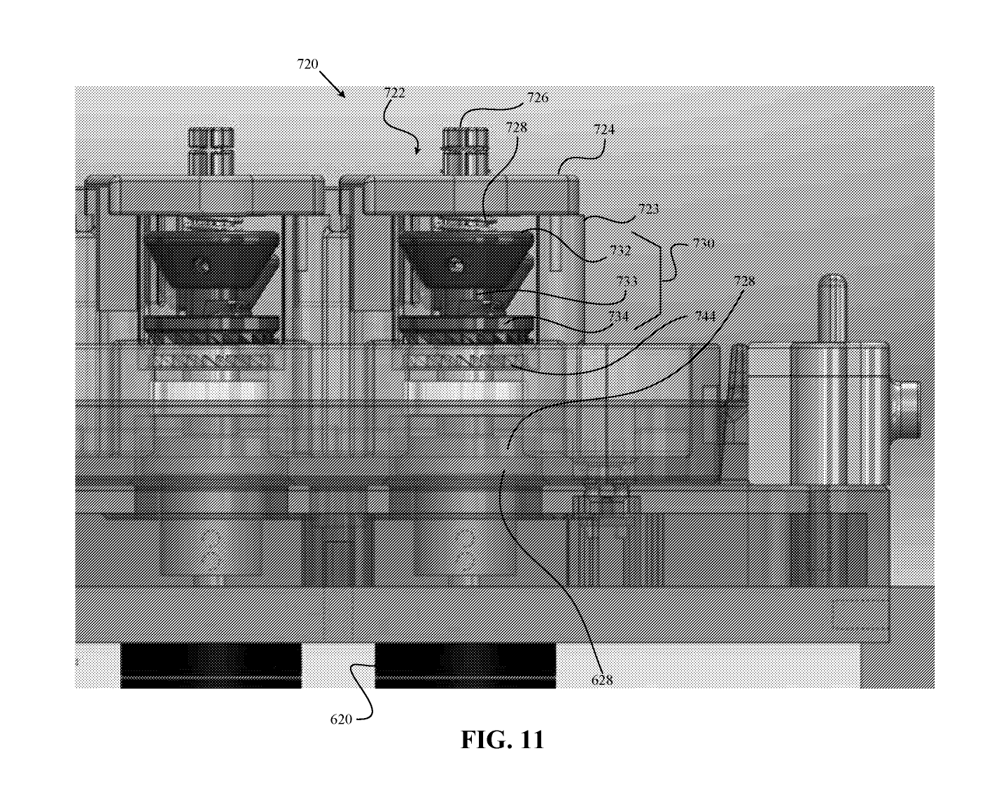

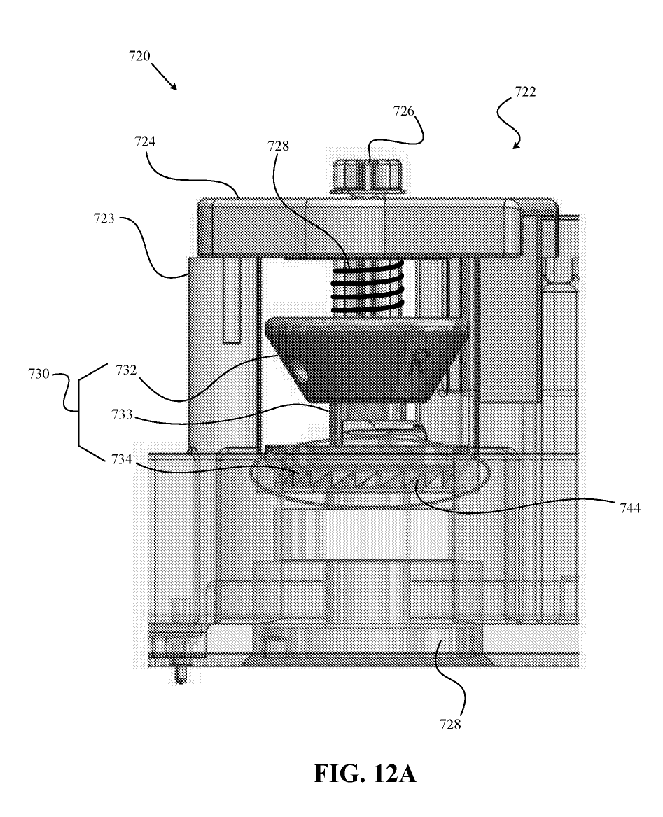

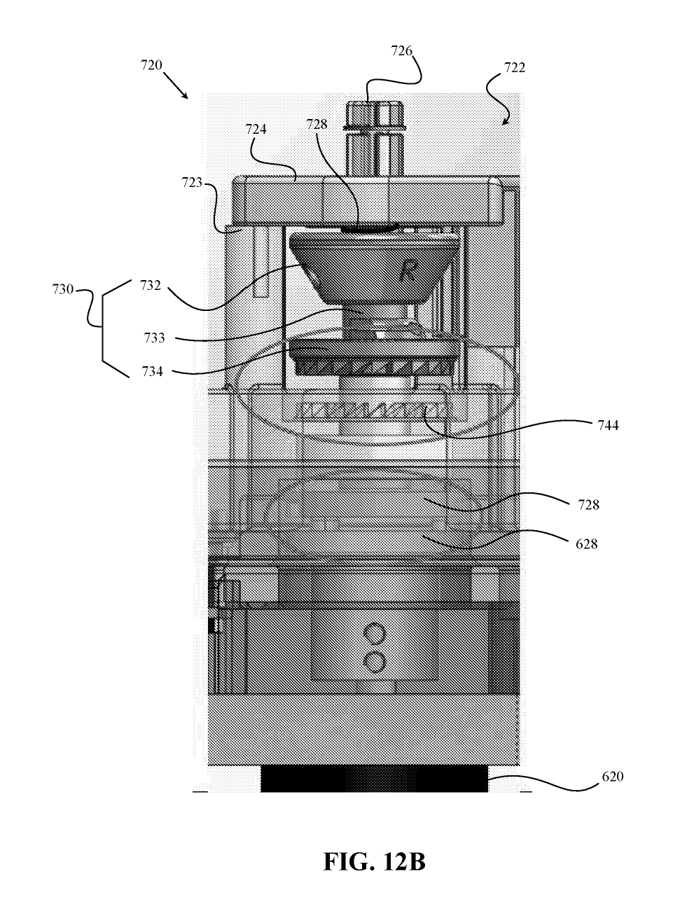

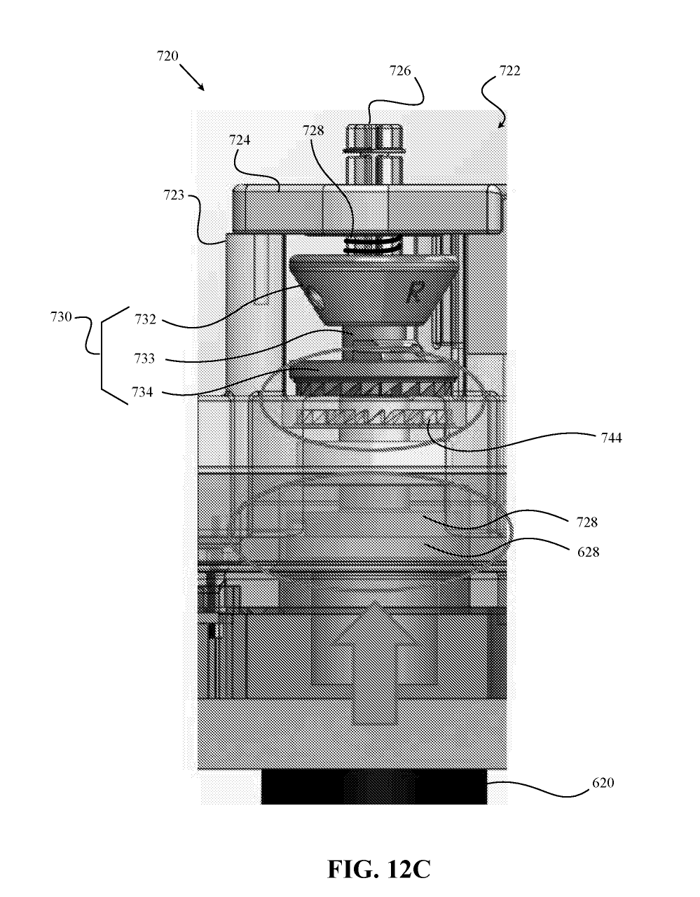

FIG. 10 is a perspective cutaway view showing representative internal portions of an actuation assembly's instrument input adapter 710 mounted to an instrument output adapter 610 of the motorbox 600 in accordance with an embodiment of the present disclosure. FIG. 11 is a corresponding cross sectional illustration showing representative internal portions of the instrument adapter 710 and instrument output adapter 610 when coupled together or matingly engaged in accordance with an embodiment of the present disclosure. FIGS. 12A-12D are cross sectional illustrations showing representative internal portions of actuation engagement structures 720 provided by the instrument input adapter 710, and the positions of elements therein, corresponding to various phases of engagement of the instrument input adapter 710 with and disengagement of the instrument input adapter 710 from the instrument output adapter 610 in accordance with an embodiment of the present disclosure.

With reference to FIG. 10, in an embodiment the instrument input adapter 710 includes a plurality of actuation engagement structures 720, such as an individual actuation engagement structure 720 for each motorbox actuator/motor 620 that is configured for controlling the robot arm/end effector 410, 420 of the particular actuation assembly 400 with which the instrument input adapter 710 is associated.

In certain embodiments, the motorbox 600 includes a single actuator/motor for controlling each DOF of the robot arm/end effector 410, 420, in which case the instrument input adapter 710 includes a single actuation engagement structure 720 corresponding to each such DOF. In such embodiments, any given DOF corresponds to a single tendon (which resides within its particular sheath).

In various embodiments, the motorbox 600 includes dual or paired actuators/motors 620 for controlling each DOF provided by the actuation assembly's robot arm/end effector 410, 420. In such embodiments, any given DOF corresponds to a pair of tendons (e.g., a first tendon that resides within a first sheath, and a second tendon that resides within a second sheath). In this case, two actuators/motors within the motorbox 600 are actuated synchronously relative to each other such that a given pair of tendons (e.g., the first tendon and the second tendon) control a given DOF of the robot arm/end effector 410, 420.

As a result, the instrument input adapter 710 correspondingly includes a pair of actuation engagement structures 720 corresponding to each robot arm/end effector DOF. In a representative implementation in which a robot arm/end effector 410, 420 are positionable/manipulable with respect to six DOFs, the motorbox 600 includes twelve actuators/motors 600a-l for controlling this robot arm/end effector 410, 420, and the instrument input adapter 710 includes twelve actuation engagement structures 720a-l. The instrument input adapter 710 mounts to the motorbox 600 such that a particular pair of actuation engagement structures 720 (e.g., actuation engagement structures 720 disposed in a side-by-side manner relative to each other along a length of the instrument input adapter 710) corresponds to and is mechanically coupled to a counterpart pair of actuators/motors 620a-l within the motorbox 600 for providing robot arm/end effector manipulability/positionability with respect to a particular robot arm/end effector DOF.

As indicated in FIG. 11 and also FIGS. 12A-12D, in an embodiment an actuation engagement structure 720 includes (a) a frame member 722 having a plurality of arm members 723 that support a frame member platform 724 that defines an upper boundary of the frame member 722, where the frame member platform 724 is perpendicular or transverse to such arm members 723; (b) an elongate input shaft 726 that extends upwardly through a center or central region of the frame member's platform 724, and downwardly toward an output disk 626 of the motorbox output adapter 610 such that it can be engaged thereby, and which is displaceable along a longitudinal axis (e.g., in a vertical direction parallel to its length); (c) a drum structure 730 mounted to and circumferentially disposed around the input shaft 726, which includes (i) a tapered drum 732 having an upper surface, an outer surface, and a bottom surface, and (ii) a first ratchet element 734 carried perpendicular or transverse to the input shaft 726 at a predetermined distance away from the bottom surface of the drum 732; (d) a resilient biasing element or spring 728 circumferentially disposed around the input shaft 726, between an underside of the frame member's platform 724 and the upper surface of the drum 732; and (e) a second ratchet element 744 perpendicular or transverse to and circumferentially disposed around the input shaft 726, and disposed below the first ratchet element 734 at a predetermined distance away from the underside of the frame member's platform 724. In various embodiments, the second ratchet element 744 is positionally fixed, immovable, or non-displaceable relative to the input shaft 726.

The drum structure includes a collar portion 733 that defines a spatial gap between the bottom surface of the drum 732 and an upper surface of the first ratchet element 734. A proximal end of a tendon can be coupled, linked, or secured to a portion of the drum structure 730 (e.g., a crimp fixture/abutment carried on an upper surface of the first ratchet element 734), and the tendon can be tightly wound around the circumference of the drum structure's collar portion 733, such that the collar portion 733 carries multiple or many tendon windings thereabout. In a direction toward its opposite/distal end, the tendon wound about the collar portion 722 can extend away from the drum structure 730, toward, into, and along the length of the actuator assembly's outer sleeve/coil 402, until reaching a given location on the actuator assembly's robotic arm 410 (e.g., at a particular position relative to a robotic arm joint or joint element) or end effector 420.

Rotation of the drum structure 730, or correspondingly rotation of the input shaft 726, results in further winding of the tendon about the drum structure's collar portion 733, or partial unwinding of the tendon from the collar portion 733, depending upon the direction in which the drum structure 730 is rotated. Winding of the tendon about the collar portion 733 results in an increase in tendon tension, and can reduce the length of the tendon that resides within the actuator assembly's outer sleeve/coil 402; and unwinding the tendon from the collar portion 733 results in a decrease in tendon tension, and can increase the length of the tendon that resides within the actuation assembly's outer sleeve/coil 402, in a manner readily understood by one having ordinary skill in the relevant art. Consequently, selective tendon winding/unwinding facilitates or enables the precise manipulation/positioning of the robotic arm/end effector 410, 420 relative to a particular DOF.

More particularly, in an embodiment providing dual motor control for each DOF, synchronous winding/unwinding of paired tendons corresponding to a specific DOF, by way of synchronous rotation of counterpart drum structures 730, results in the manipulation/positioning of the robotic arm/end effector 410, 420 in accordance with this DOF. Such synchronous drum structure rotation can selectively/selectably occur by way of a pair of actuator/motors 620 and corresponding output disks 626 to which actuation engagement structure input shafts 726 can be rotationally coupled, as further detailed below.

When the instrument input adapter 710 is not engaged with or has been disengaged from the instrument output adapter 610 of the motorbox 600, an actuation engagement structure's spring 728 biases or pushes the actuation engagement structure's drum structure 730 downward to a first or default position, such that the first ratchet element 734 securely matingly engages with the second ratchet element 744. Such engagement of the first ratchet element 734 with the second ratchet element 744 when the spring 728 biases the drum structure downward 730 is illustrated in FIG. 12 A. As a result of such engagement of the first and second ratchet elements 734, 744, the drum structure 730 is prevented from rotating, and thus the tension in the tendon corresponding to the drum structure 730 is maintained or preserved (e.g., the tension in the tendon cannot change or appreciably change).

As indicated above, the actuation engagement structure's input shaft 726 is displaceable parallel to or along its longitudinal axis. As the instrument input adapter 710 is mounted or installed onto the instrument output adapter 610 of the motorbox 600 (e.g., by way of one or more snap-fit couplings), a bottom surface of a lower plate 728 carried by the input shaft 726 below the second ratchet element 744 contacts a set of projections carried by an upper surface of an output disk 628 associated with a particular actuator/motor 620. Consequently, the spring 728 is compressed, and the input shaft 726 and the drum structure 730 carried thereby are upwardly displaced such that the distance between the upper surface of the drum 732 and the underside of the frame member's platform 724 decreases, as indicated in FIG. 12B. Such upward displacement of the drum structure 730 causes the first ratchet element 734 to disengage from the second ratchet element 744. This can correspond to a situation in which the instrument input adapter 710 is installed or mounted on the instrument output adapter of the motorbox 600, but the input shaft 726 is not yet rotationally rotatably/rotationally coupled to with the output disk 626 of the actuator/motor 620.

During the mounting of the instrument input adapter 710 onto the instrument output adapter 610 of the motorbox 600, or once the instrument input adapter 710 is fully/securely mounted onto the instrument output adapter 610 (e.g., as can be detected by way of a set of sensors), corresponding to a situation in which the input shaft 726 and drum structure 730 have been vertically displaced upward and the first and second ratchet elements have become disengaged from each other, the actuators/motors 620 within the motorbox 600 commence an initialization process (e.g., under the direction of the control unit 800). During the initialization process, each actuator/motor 620 rotates its corresponding output disk 628 until the set of projections carried by the output disk 628 catch or matingly engage with counterpart recesses within the bottom surface of the input shaft's lower plate 728.

Once the projections carried by the output disk 628 catch or matingly engage with counterpart recesses formed in the input shaft's lower plate 728, the input shaft 726 is rotationally coupled to an intended actuator/motor 620, in a manner illustrated in FIG. 12C. When such output disk projections and lower plate recesses are rotationally coupled, the actuator/motor 620 can selectively precisely control the winding and unwinding of the tendon about the collar portion 733 of the drum structure 730, and/or precisely control tendon tension, to thereby manipulate/position the robotic arm/end effector 410, 420 in an intended manner in response to surgeon input received at the master station 100.

When the instrument input adapter 710 is disengaged, dismounted, or detached from the instrument output adapter 610, decompression of the spring 728 pushes the upper surface of the drum structure 730 downward, such that the first ratchet element 734 matingly engages with the second ratchet element 744 in a manner illustrated in FIG. 12D. Rotation of the input shaft 726 and the disc structure 730 are then prevented, and tendon tension is thus maintained in a manner essentially identical or analogous to that described above in relation to FIG. 12A.

In alternate embodiments, the first and second ratchet elements 734, 744 can be replaced by or implemented as first and second friction plates 734, 744, or other types of securely engageable/releasable structures (e.g., discs having counterpart male and female engagement elements) configured for reliably maintaining or preserving tension tendon when engaged (e.g., reliably preventing tendon winding/unwinding relative to the input shaft's longitudinal axis until disengaged). Such first and second elements 734, 744 configured for reliably maintaining or preserving tendon tension when engaged can thus be referred to as tendon tension maintenance elements or antirotation elements.

### Representative Alternate Docking Station/Translation Unit Configurations

The translation unit 510 carried by or incorporated into the docking station 500 enables longitudinal/surge displacement of each actuation assembly 400a,b and the imaging endoscope assembly 450 (e.g., on an individual basis). In embodiments described above, the translation unit 510 includes receivers or clips 530a-c configured for mating engagement with corresponding collars 430a-c carried by the outer sleeves 402a-c of the actuation assemblies 400a,b or the imaging endoscope assembly 450. Additionally, the aforementioned instrument input adapters 710 and the imaging input adapter 750, as well as the instrument output adapters 610 and the imaging output adapter 650 of the motorbox 600, are located away from the docking station 500.

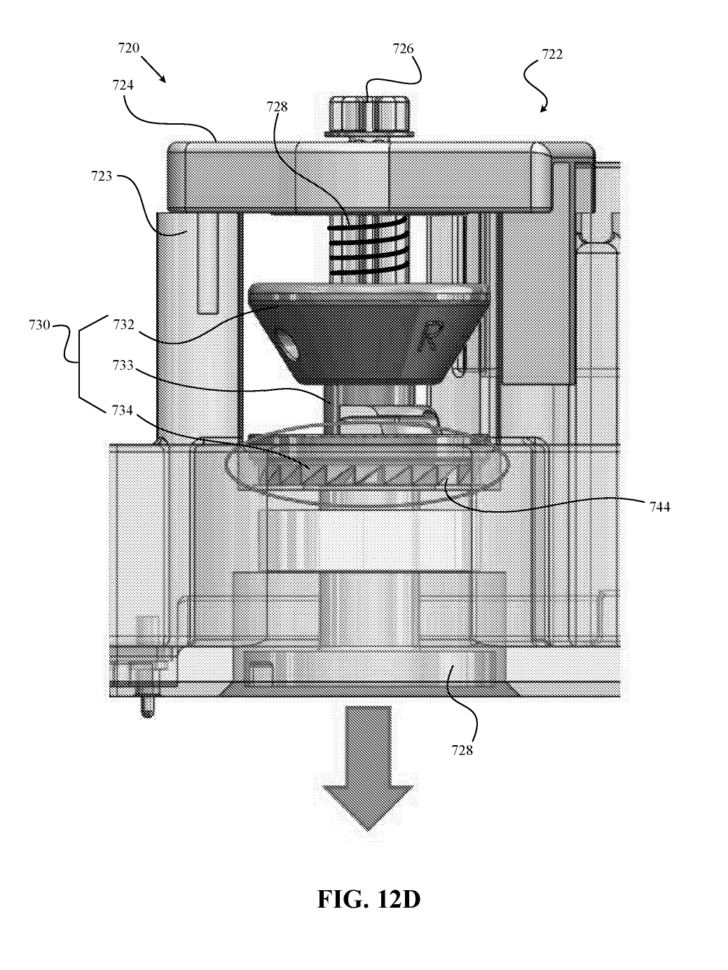

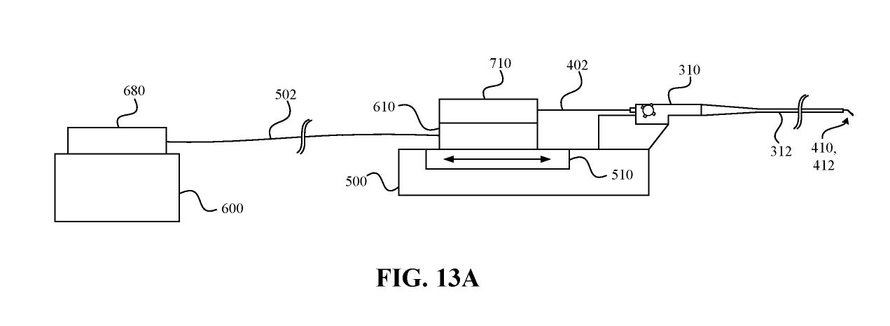

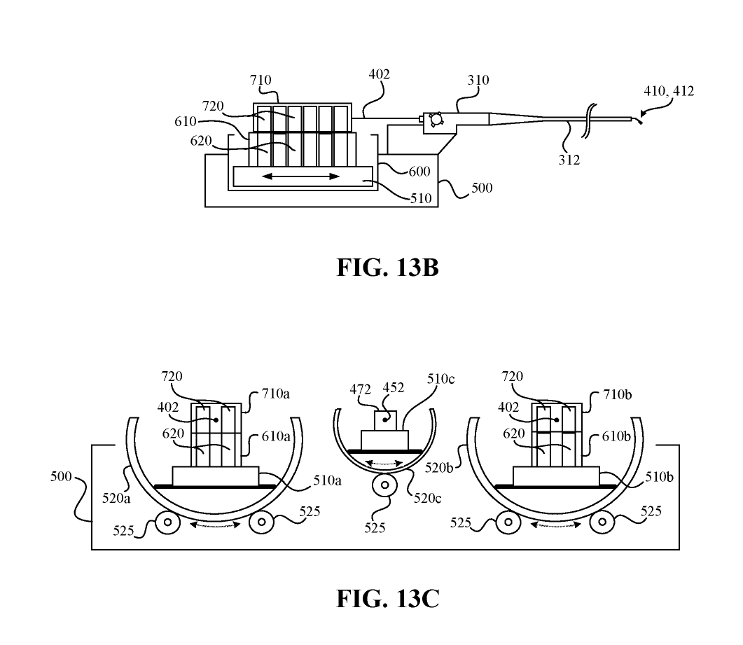

FIG. 13A illustrates an alternate embodiment of a docking station 500 in accordance with the present disclosure, in which the docking station 500 and its translation unit 510 are configured for carrying a set of instrument output adapters 610 and an imaging output adapter 650, onto which the instrument input adapters 710 and the imaging input adapter 750 can be mounted or installed. In such an embodiment, actuation stages of the translation unit 510 can independently proximally-distally displace each instrument output adapter 610, and hence each instrument input adapter 710 coupled thereto; as well as the imaging output adapter 650, and hence the imaging input adapter 750 coupled thereto, such that the robotic arms/end effectors 410a,b, 420a,b and the imaging endoscope 460 can be correspondingly longitudinally displaced/surged. In some embodiments, each instrument output adapter 610 and the imaging output adapter 650 can be coupled to the motorbox 600 by way of a set of tethers 502, for instance, which is coupled to or linked with a set of additional or secondary output adapter structures 680 carried by the motorbox 600. Each tether 502 includes or carries therein a set of tendons configured for transferring mechanical forces, as will be understood by an individual having ordinary skill in the relevant art in view of the description herein.

FIG. 13B illustrates yet another embodiment of a docking station 500 in accordance with the present disclosure, in which the docking station 500 is configured for carrying the motorbox 600, and the translation unit 510 is configured for proximally-distally displacing individual sets of actuators/motors 620 within the motorbox 600 (e.g., displacing the actuators/motors 620 corresponding to a particular or selected individual actuation assembly 400), along with each instrument output adapter 610 and instrument input adapter 710 coupled thereto, plus the imaging output adapter 650 and the imaging input adapter 750 if present, to independently longitudinally displace/surge each robotic arms/end effector 410a,b, 420a,b and the imaging endoscope 460.

Thus, in embodiments such as that shown in FIG. 13B, the translation unit 510 carries the actuators/motors 620 to which each instrument input adapter 710 and the imaging input adapter 750 are couplable/coupled, where such actuators/motors 620 are configured for enabling selective non-surge spatial positioning/manipulation of each robotic arm 410a,b and its corresponding end effector 420a,b and in those embodiments that support it, selective non-surge spatial positioning/manipulation of the imaging endoscope 460 during an endoscopic procedure. The translation unit 510 is configured for selectively displacing particular sets or subsets of actuators/motors 620 (and correspondingly, the instrument adapter 710 or the imaging input adapter 750 engaged therewith) to thereby longitudinally displace/surge a given robotic arm/end effector 410a,b, 420a,b within or across a maximum surge displacement distance (e.g., up to approximately 10-15 cm). The actuators/motors 620 corresponding to each robotic arm/end effector 410a,b, 420a,b can be carried and selectively translated to effectuate robotic arm/end effector surge displacement by way of an associated linear translation stage, mechanism, or device of the translation unit 510, such as a ball screw or linear actuator. Similarly, the actuators/motors 620 corresponding to the imaging endoscope 460 can be carried and selectively translated to effectuate imaging endoscope surge displacement by another linear translation stage, mechanism, or device of the translation unit 510, such as a ball screw or linear actuator.

Embodiments such as those shown in FIG. 13A-13B can reduce an amount of tendon backlash, and hence can more precisely maintain an intended/predictable level or range of tendon tension, by way of shortening the distance between each tendon's distal end and an actuator/motor 620 within the motorbox 600. An embodiment such as that shown in FIG. 13B can result in a system 10 having highly consistent/predictable tendon tension levels/ranges, and significantly reduced or minimized/minimum tendon backlash.

In some embodiments, in addition to carrying a set of surge displacement/proximal-distal translation mechanisms 500, the docking station 500 is also configured for carrying a set of mechanisms or devices by which some or each actuation assembly 400a,b and/or the imaging endoscope 460 can be selectively individually rotated about their longitudinal or central axes, to thereby respectively enable selective individual roll motion of the actuation assemblies 400a,b and/or the imaging endoscope 460. In such embodiments, the actuation assemblies 400a,b and/or the imaging endoscope assembly 450 need not include internal roll motion mechanisms themselves (e.g., one or more internal roll joints). Rather, roll motion is providable/provided to the actuation assemblies 400a,b and/or the imaging endoscope 460 by way of mechanisms or devices that are external to the actuation assemblies 400a,b and/or the imaging endoscope 460, respectively.

As a representative example, FIG. 13C provides a cross-sectional front view through portions of a docking station 500 configured for carrying a set of cradle or drum structures 520a-c that are rotatably coupled to or engaged with corresponding roll motion actuators/motors 525a,c and/or precision discs, rollers, or gears associated therewith, by which roll motion is individually providable to each actuation assembly 400a,b and the imaging endoscope 460. In the embodiment shown, a first cradle 520a carries a first translation mechanism 510a (e.g., a linear actuator) configured for selectively providing surge displacement/proximal-distal translation to a first actuation assembly 400a such as that shown in FIG. 4B, including a first instrument adapter 710a corresponding thereto. More particularly, the first translation mechanism 510a carries a first instrument output adapter 610a (and the actuators 620 thereof), to which the first instrument input adapter 710a (and the actuation engagement structures 720 thereof) is engageable/engaged, in a manner identical or analogous to that previously described.

The first cradle 520a is rotatably coupled or engaged with a first roll motion actuator 525a and possibly a set of associated roll motion discs, rollers, and/or gears by which the first cradle 520a can be precisely rotated across a predetermined angular range, for instance, +/−180 degrees in response to actuation of the first roll motion actuator 525a. An axis of rotation of the first cradle 520a is parallel to an axis along which surge displacement is providable to the first actuation assembly 400a, and an axis along which the outer sleeve 402 of the first actuation assembly 400a interfaces with the first instrument adapter 710a.

Similarly, a second cradle 520b carries a second translation mechanism 520b configured for selectively providing surge displacement/proximal-distal translation to a second actuation assembly 400b such as that shown in FIG. 4C, including a second instrument adapter 710b corresponding thereto. More particularly, the second translation mechanism 510b carries a second instrument output adapter 610b (and the actuators 620 thereof), to which the second instrument input adapter 710b (and the actuation engagement structures 720 thereof) is engageable/engaged, in a manner identical or analogous to that described above. The second cradle 520b is rotatably coupled or engaged with a second roll motion actuator 525b and possibly a set of associated roll motion discs, rollers, and/or gears by which the second cradle 520b can be precisely rotated across a predetermined angular range (e.g., +/−180 degrees), in a manner identical or analogous to that described above. An axis of rotation of the second cradle 520b is parallel to an axis along which surge displacement is providable to the second actuation assembly 400b, and an axis along which the outer sleeve 402 of the second actuation assembly 400b interfaces with the second instrument adapter 710b.

Finally a third cradle 520c carries a third translation mechanism 510c configured for providing surge displacement/proximal-distal translation to an imaging endoscope 460, such as an imaging endoscope 460 that excludes or omits tendons or other types of internal control elements for controlling or providing heave, sway, and/or pitch motion. A proximal end of the imaging endoscope 460 can be coupled to an imaging translation adapter 472 that detachably interfaces or engages with the third translation mechanism 510c, and by which electronic and/or optical elements of the imaging endoscope 460 are couplable/coupled to the imaging subsystem 210. The third cradle 520c is rotatably coupled or engaged with a third roll motion actuator 525c and possibly a set of associated roll motion discs, rollers, and/or gears by which the third cradle 520c can be precisely rotated across a predetermined angular range (e.g., +/−180 degrees), in a manner identical or analogous to that described above. An axis of rotation of the third cradle 520c is parallel to an axis along which surge displacement is providable to the imaging endoscope 460, and an axis along which the outer sleeve 452 of the imaging endoscope 460 interfaces with the imaging translation adapter 472.

Depending upon embodiment details, the first, second, and third roll motion actuators 525a-c can be individually actuated in response to control signals generated by the master system 100 and/or a set of controls carried by the transport endoscope body 310.

### Representative Aspects of Tendon Pretensioning/Retensioning

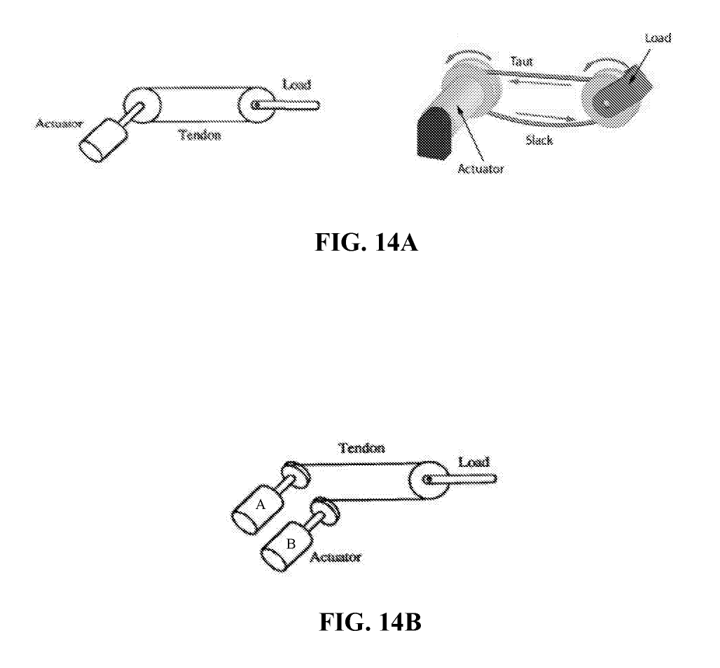

FIG. 14A illustrates a representative single actuator/motor per DOF configuration, and potential backlash-like effects that can be associated therewith. FIG. 14B illustrates a representative dual actuator/motor per DOF configuration in accordance with an embodiment of the present disclosure. As indicated in FIG. 14B, when two actuators/motors are used for controlling each robotic arm/end effector DOF, undesirable/unwanted tendon slack and backlash-like effects can be reduced (e.g., significantly reduced).

Each tendon resides within a corresponding sheath. Appropriate and precise tendon pretensioning ensures that tendons can be controlled in a more precise and repeatable manner during an endoscopic procedure. In various embodiments, the sheath exhibits a coil structure (e.g., a helical coil structure), and hence the sheath has spring or spring-like properties. Interactions between a tendon and its corresponding sheath (e.g., as a result of tendon-sheath friction) cannot be reliably predicted in the absence of knowledge of the tortuosity of a path along which the tendon and its surrounding sheath are routed. Thus, the tension to which any given tendon is subjected immediately prior to the initiation of an endoscopic procedure depends upon the tortuosity of a path along which the tendon and its corresponding sheath are routed for purpose of carrying out the procedure.

Unlike earlier master-slave flexible robotic endoscopy systems, a system in accordance with an embodiment of the present disclosure need not establish and maintain precise tendon tensions from the time of actuation assembly manufacture onward. Rather, in various embodiments, an initial minimum acceptable tendon pretension level or range can be established as part of manufacturing an actuator assembly 400 (e.g., approximately 1.0-30.0 N, depending upon tendon length), and precise tendon pretensioning or retensioning can occur by way of adjustment of actuator/motor position and/or torque prior to the performance of an endoscopic procedure.

Depending upon embodiment details, tendon pretensioning can occur by way of a fixed pretensioning technique involving the application of fixed or predetermined motor parameters (e.g., torque parameters), or an active/dynamic pretensioning technique involving on-the-fly determination of motor torque parameters, such that a correct or approximately correct amount of tension can be applied to the tendons prior to the initiation of an endoscopic procedure, or possibly during an endoscopic procedure.

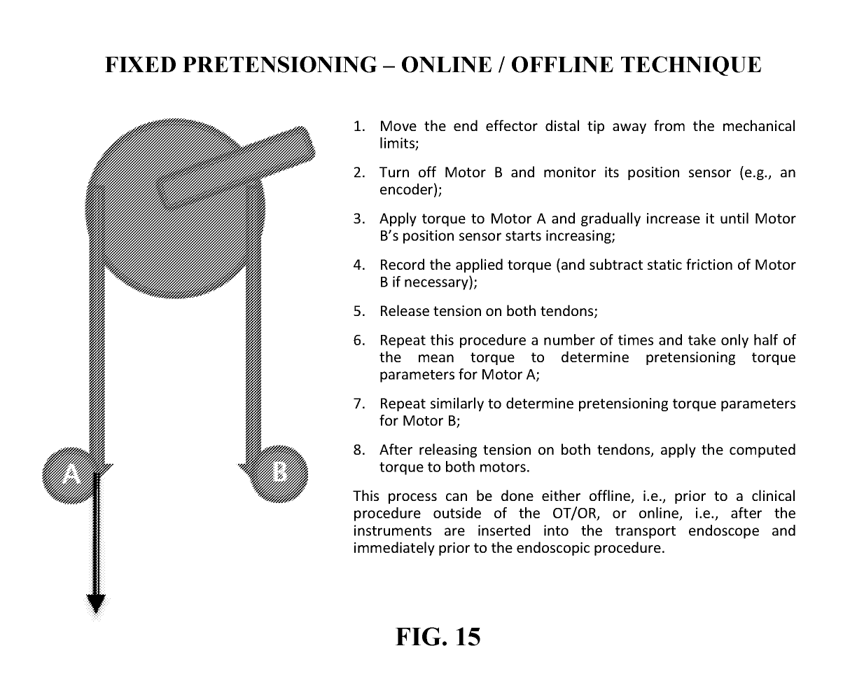

FIG. 15 is an illustration showing portions of a representative offline/online fixed pretensioning technique, procedure, or process in accordance with an embodiment of the present disclosure. This procedure can be performed either “offline,” i.e., prior to a clinical procedure and outside of the OT/OR; or “online,” i.e., in the OT/OR after the actuation assemblies 400 are inserted into the flexible elongate shaft 312 and the robotic arms 410a,b and end effectors 420a,b are disposed at the distal end thereof, immediately prior to performance of an endoscopy procedure.

In several embodiments, for a given pair of actuators 620 defined by Actuator A and Actuator B controlling a particular DOF of a selected robotic arm 410a/410b and its end effector 420a/420b by way of a pair of tendons (e.g., tendon A corresponding to Actuator A, and tendon B corresponding to Actuator B), the fixed pretensioning technique involves the following sequence of actions, operations, or steps:
1. Move the distal tip of the end effector 420a/420b away from the mechanical limits of Actuator B.
2. Turn off Actuator B and begin monitoring a position sensor corresponding Actuator B (e.g., an encoder of Actuator B).
3. Apply torque to Actuator A and gradually increase the torque applied to Actuator A until the position sensor of Actuator B indicates the position of Actuator B is changing.
4. Record the applied torque of Actuator A, and subtract the static friction of Actuator B if necessary.
5. Release tension on both tendons (i.e., tendons A and B).
6. Repeat steps 1-5 one or more times (e.g., 2-10 or more times), and take one-half of the mean recorded applied torque of Actuator A to determine or define pretensioning torque parameters for Actuator A.
7. Correspondingly repeat steps 1-6 for actuator B, while Actuator A is off.
8. After determining the pretensioning torque parameters for Actuator A and Actuator B, release tension on both tendons (i.e., tendons A and B), and apply torque to Actuator A using the computed pretensioning torque parameters for Actuator A, and apply torque to Actuator B using the computed pretensioning torque parameters for Actuator B.

In an embodiment, the offline fixed pretensioning technique involves running preliminary experiments under various representative tortuosity configurations; measuring actuator/motor torque values corresponding to such representative tendon/sheath tortuosity configurations; averaging measured torque values corresponding to one or more tortuosity configurations; and storing (e.g., in a memory or on a data storage device) one or more sets of averaged torque values corresponding to particular tortuosity configurations. Depending upon the nature of an endoscopic procedure under consideration, and an expected tendon/sheath tortuosity associated therewith, an appropriate set of averaged torque values can be retrieved (e.g., from memory or a data storage medium), and applied to the tendons within an actuation assembly 400 by way of the actuators/motors 620 to which the actuation assembly 400 is coupled just prior to commencement of the endoscopic procedure. Such a technique can be also applied online or on the fly, i.e., immediately prior to the endoscopic procedure. In the online case, the tortuosity of the path is already set, so the pretensioning is optimized for this specific path.

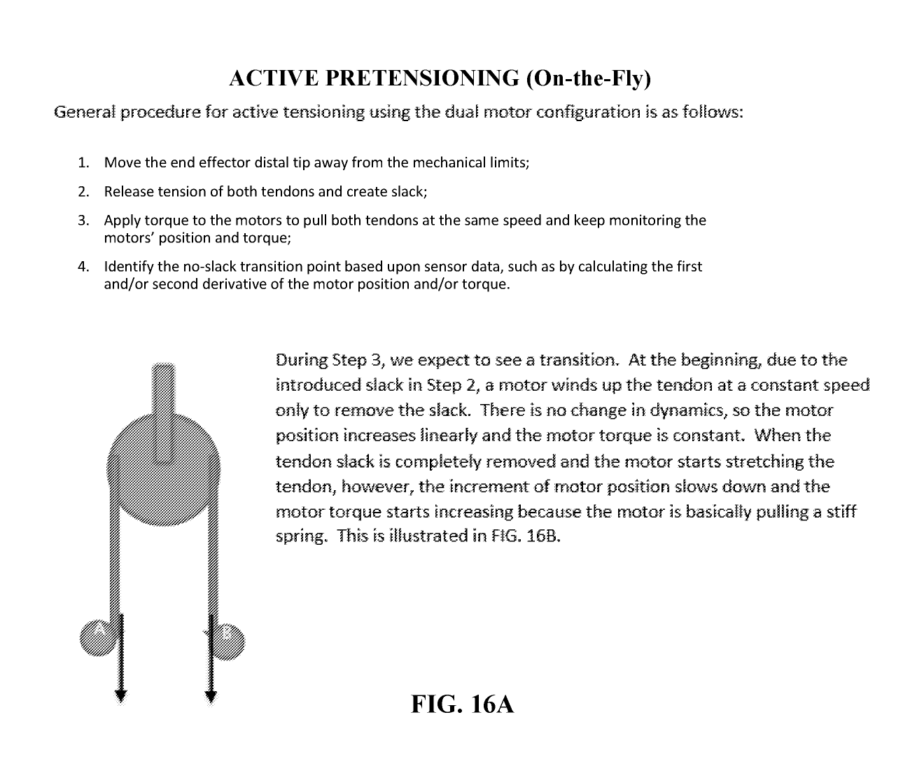

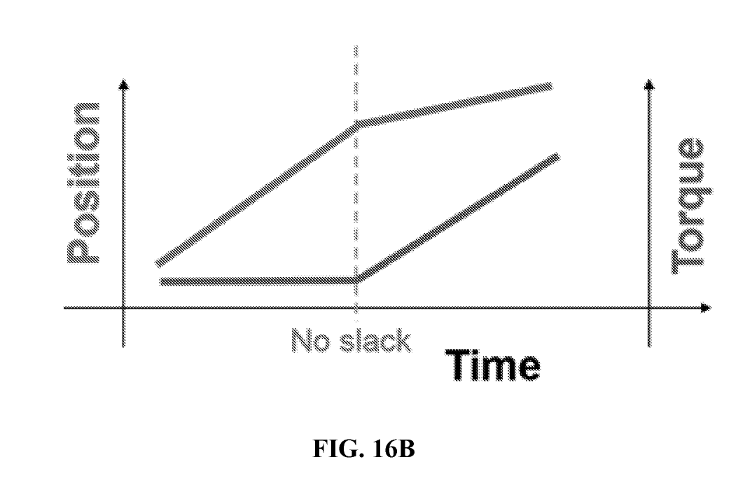

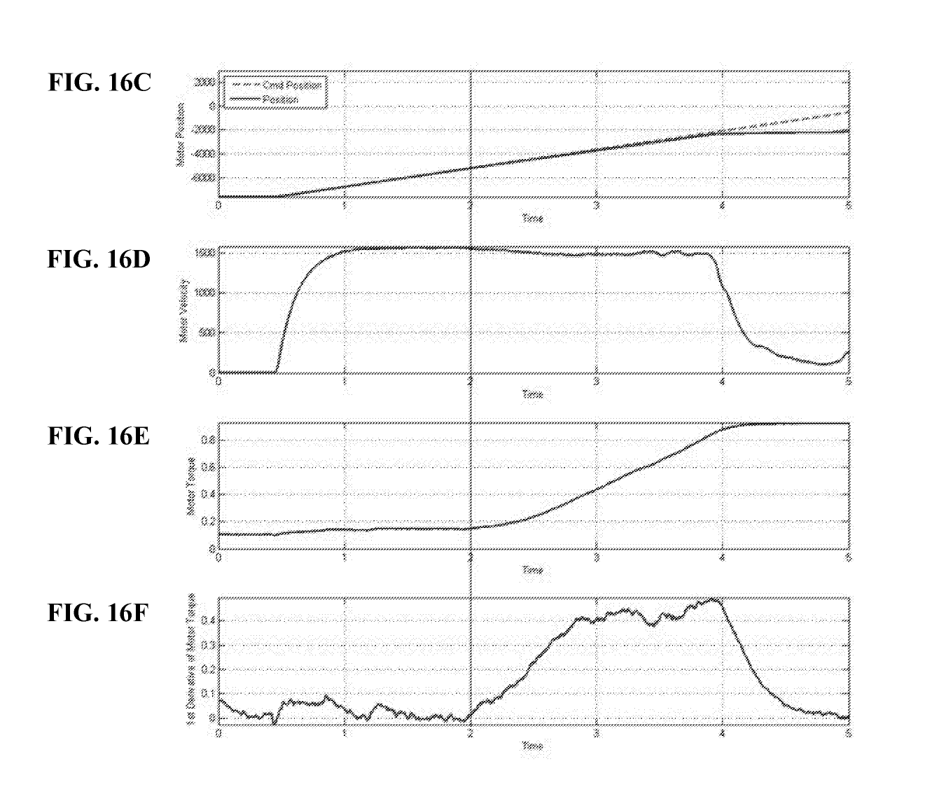

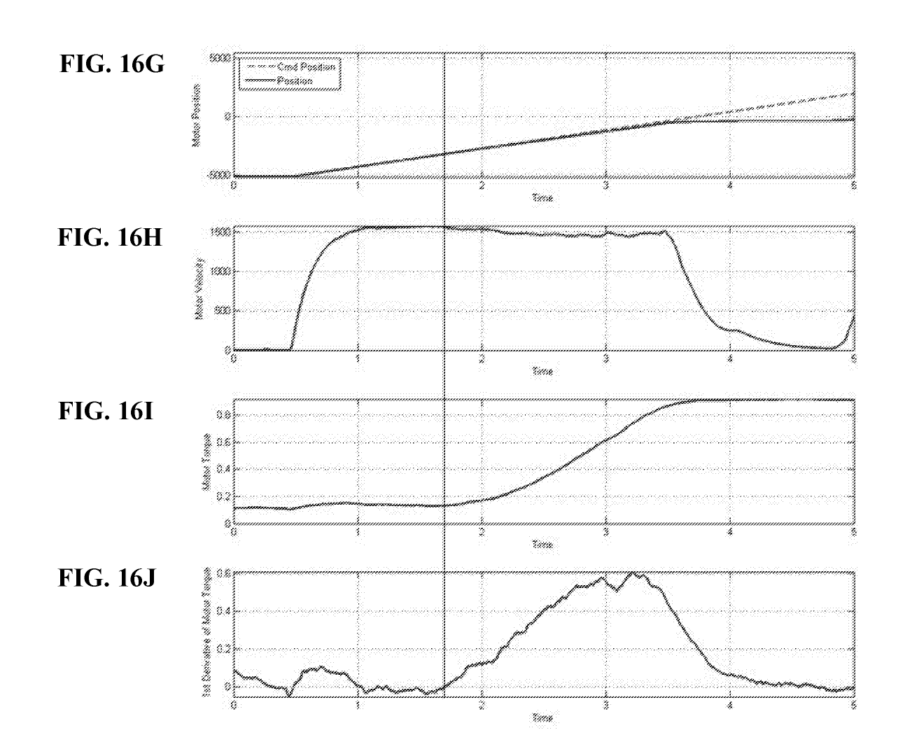

FIG. 16A is an illustration of portions of an active pretensioning/retensioning technique, procedure, or process in accordance with an embodiment of the present disclosure, and FIG. 16B is a representative graph of actuator/motor position and torque corresponding thereto. The active pretensioning technique involves determining a no-slack transition point, such as by way of calculating a first and/or a second derivative of a measured tension profile or curve. For a given tendon, the no-slack transition point can be automatically identified in real time, and an appropriate pretension or retension can be applied to the tendon. The active pretensioning/retensioning technique can be performed in the OT/OR after the actuation assemblies 400 are inserted into the flexible elongate shaft 312 and the robotic arms 410a,b and end effectors 420a,b are disposed at the distal end thereof, immediately prior to performance of an endoscopy procedure; or during the endoscopy procedure. Applying the correct amount of tension is important to ensure efficient proximal-to-distal force transmission. If the applied tension is too small, tendon slack exists, which can create backlash-like effects. If the applied tension is too large, it increases friction between the tendon and the sheath, which can also create backlash-like effects.

In several embodiments, for a given pair of actuators 620 defined by Actuator A and Actuator B controlling a particular DOF of a selected robotic arm 410a/410b and its end effector 420a/420b by way of a pair of tendons (e.g., tendon A corresponding to Actuator A, and tendon B corresponding to Actuator B), the active pretensioning technique involves the following sequence of actions, operations, or steps:
1. Move the distal tip of the end effector 420a/420b away from its mechanical limits.
2. Release tension on both tendons (i.e., tendons A and B) and create slack therein.
3. Apply torque to Actuator A and Actuator B simultaneously to pull both tendons (i.e., tendons A and B) at the same speed while monitoring the position and torque of Actuator A and the position and torque of Actuator B.
4. Identify the no-slack transition point for each of Actuator A and Actuator B based on sensor data, such as by calculating the first and/or second derivative of the monitored position and/or torque of Actuator A and Actuator B.
5. Simultaneously (i) establish the pretension of tendon A by applying torque to Actuator A at the torque level corresponding to or defined by the no-slack transition point determined for Actuator A, and (ii) establish the pretension of tendon B by applying torque to Actuator B at the torque level corresponding to or defined by the no-slack transition point determined for Actuator B.

The active pretensioning procedure can be repeated a number of times (e.g., 2-10 or more times) in order to obtain a mean no-slack transition point for Actuator A and a mean no-slack transition point for Actuator B, in a manner readily understood by an individual having ordinary skill in the relevant art.

FIGS. 16C-16F are graphs or plots respectively indicating measured motor position, measured motor velocity, measured motor torque, and the first derivative of measured motor torque for a first actuator/motor (e.g., Motor A) of a particular actuator/motor pair with respect to time during while performing the active pretensioning technique of FIG. 16A. FIG. 16G-16J are graphs or plots respectively indicating measured motor position, measured motor velocity, measured motor torque, and the first derivative of measured motor torque for a second actuator/motor (e.g., Motor B) of the actuator/motor pair under consideration with respect to time during while performing the active pretensioning technique of FIG. 16A. In both sets of plots, signals are normalized and/or filtered as necessary to process data effectively.

For Motor A, the no-slack transition point occurred at T=2.0, corresponding to decreased motor A velocity and the generation of corresponding position error. This is apparent from the plots of the measured motor torque and the first derivative of the measured motor torque. For Motor B, the no-slack transition point occurred at T=1.7. For both Motor A and Motor B, analogous or similar features can be identified from sequences of measured values or plots thereof. A large transition occurred at T=3.9 in FIGS. 16C-16F, and at T=3.5 in FIGS. 16G-16J. This large transition was due to saturated motor torque, and is not related to the no-slack transition point of each actuator/motor. Each no-slack transition point can be automatically identified under program instruction control, such as by way of processing unit execution of one or more algorithms (e.g., corresponding to program instruction sets stored in memory or other computer readable medium) associated with signal processing, statistical analysis, and/or machine learning, among others. One or more of such algorithms can be executed multiple times to identify a no-slack transition point more accurately.

### Representative Pulley-Based Roll Joint Primitive and Crimp-Free Tendon Anchors

In some embodiments, a robotic arm 410 can include a roll joint or roll joint primitive, by which one or more portions of the robotic arm can be rotated or rolled about a central or longitudinal axis of the roll joint/roll joint primitive. It is desirable in a roll joint/roll joint primitive that can reduce or minimize tendon wear due to friction/abrasion associated with tendon actuation of the roll joint/joint primitive. In various embodiments, such as those corresponding to surgical procedures, is further desirable to minimize the amount of space occupied by a roll joint/roll joint primitive.

Certain axes of a robotic surgical instrument have size constraints that can discourage or prevent the use of a conventional/traditional tendon crimp termination for anchoring a tendon to an actuated element. In some embodiments in accordance with the present disclosure, a roll joint/roll joint primitive excludes conventional/traditional tendon crimp terminations for anchoring a tendon to the roll joint/roll joint primitive. Rather, a tendon actuated element such as a roll joint/roll joint primitive in accordance with an embodiment of the present disclosure can include a crimp-free tendon anchoring structure, which provides tendon anchoring by way of frictional forces along (a) a winding or tortuous path or channel through which the tendon travels, and/or (b) a tendon path through the thickness of the actuated element itself (e.g., from a first or outer side of the actuated element, into and through the thickness of the actuated element to a second or inner side of the actuated element, and back through the thickness of the actuated element to the first/outer side of the actuated element).

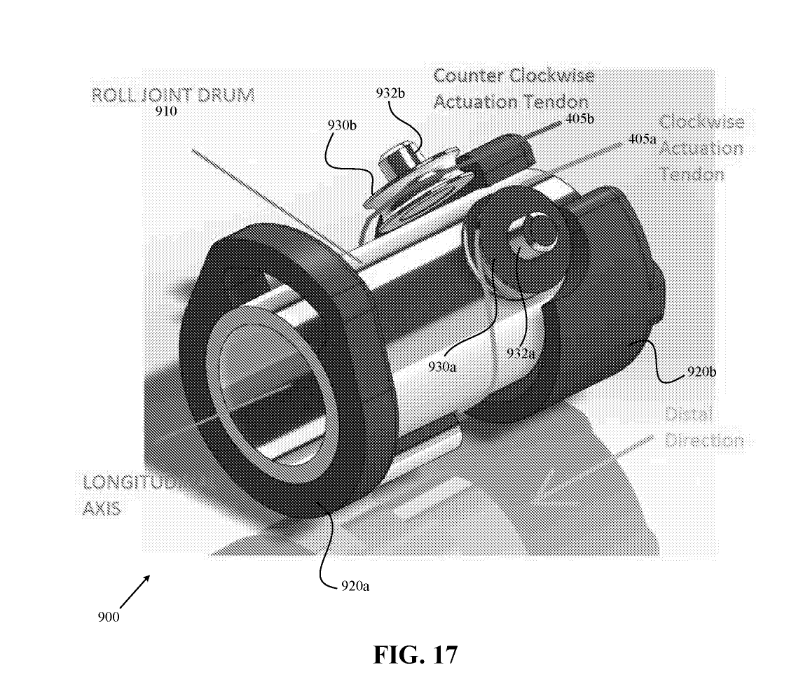

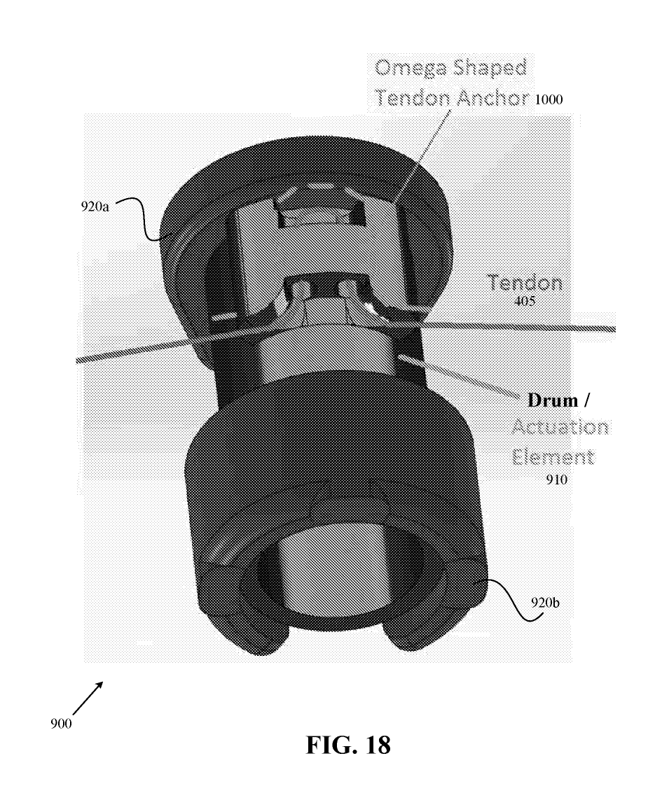

FIGS. 17 and 18 are schematic illustrations showing portions of a crimp-free pulley-based roll joint or roll joint primitive 900 in accordance with an embodiment of the present disclosure, which can reduce or minimize tendon wear resulting from friction/abrasion, and which can reduce/minimize a spatial volume required for operation of the roll joint/roll joint primitive 900. In an embodiment, the roll joint primitive 900 includes a barrel, barrel structure, drum, or drum structure 910 having a central or longitudinal axis therethrough; a set of collars 920a,b configured for carrying the drum 910; and a plurality of pulleys 930a,b such as a clockwise actuation pulley 930a and a counterclockwise actuation pulley 430b disposed above an outer surface of the drum 910, around which a clockwise actuation tendon 405a and a counterclockwise actuation tendon 405b can respectively travel such that the roll joint primitive 900 can be correspondingly rotated in a clockwise or counterclockwise direction about its central/longitudinal axis. The pulleys 930 can be supported away from the outer surface of the drum 910 by a set of arm members (not shown) that receive a central shaft 932a,b corresponding to each pulley 930, and which extend between a first collar 920a and a second collar 920b, in a manner readily understood by one having ordinary skill in the relevant art. The outer surface of the drum 910 is a smooth, non-abrasive, polished, and/or lubricated surface; and an inner surface of each collar 920a,b is a low-friction surface.

FIG. 18 illustrates a crimp-free tendon anchoring element 1000 in accordance with an embodiment of the present disclosure. In an embodiment, the crimp-free tendon anchoring element 1000 can be carried by or formed in a given actuation element such as a roll joint drum 910, and includes at least one omega shaped or U-shaped segment that provides a corresponding omega-shaped and/or U-shaped channel, passage, or groove through which a given tendon 405 can be routed. A crimp-free tendon anchoring element 1000 in accordance with an embodiment of the present disclosure, such as the omega-shaped crimp-free tendon anchoring element 1000 shown in FIG. 17, includes a multi-curved/multi-bend, winding, and/or tortuous tendon pathway providing sufficient friction points for preventing tendon slippage in response to increasing or changing tendon tension. That is, a crimp-free tendon anchoring element in accordance with an embodiment of the present disclosure exhibits an overall static friction level that is sufficiently or significantly higher than applied tendon actuation forces, such that the tendon slippage during tendon actuation is avoided, and the tendon 405 is effectively anchored in place without or in the absence of a conventional tendon crimp element. In certain embodiments, a crimp-free tendon anchoring element 1000 can additionally include one or more regions, sections, or lengths in or along which an adhesive secures outer surfaces of the tendon 405 to inner surfaces of the tendon anchoring element.

In addition or as an alternative to the foregoing, a crimp-free tendon anchoring element can include a plurality of openings or “eyelets” through an actuated element, into and through which a given tendon 405 can be routed such that the tendon 405 is disposed on or runs along/across both an outer surface/side of the actuated element and an inner surface of the actuated element.

Aspects of particular embodiments of the present disclosure address at least one aspect, problem, limitation, and/or disadvantage associated with exiting master-slave flexible robotic endoscopy systems and devices. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed systems, components, processes, or alternatives thereof, may be desirably combined into other different systems, components, processes, and/or applications. In addition, various modifications, alterations, and/or improvements may be made to various embodiments that are disclosed by a person of ordinary skill in the art within the scope of the present disclosure.

1. A master-slave endoscopy system comprising:
an endoscope having a main body from which a flexible elongate shaft extends, the flexible elongate shaft spanning a length between a proximal end and a distal end thereof, the flexible elongate shaft having a plurality of channels disposed therein along its length including a first channel, a second channel, and a third channel;
a robotically driven actuation assembly removably inserted into the first channel, the robotically driven actuation assembly comprising:
a robotic arm having a robotically driven end effector coupled thereto; and
a second plurality of tendons operable for spatially manipulating the robotic arm and its end effector in response to forces applied thereto;
an imaging endoscope removably inserted into the second channel; and
a manually driven actuation assembly removably inserted into the third channel, the manually driven actuation assembly having a manually operated endoscopic instrument coupled thereto.

2. The system of claim 1, further comprising:
a first set of actuators couplable to the robotically driven actuation assembly, and configured for applying forces to the second plurality of tendons thereof.

3. The system of claim 2, wherein the imaging endoscope comprises a portion of an imaging endoscope assembly comprising an adapter by which the imaging endoscope is couplable to an actuator configured for providing surge displacement to the imaging endoscope.

4. The system of claim 3, wherein the imaging endoscope assembly further comprises a plurality of tendons carried therein coupled by way of the adapter to a second set of actuators configured for providing the imaging endoscope with at least one of heave, sway, and pitch motion.

5. (canceled)

6. The system of claim 2, wherein the robotically driven actuation assembly is configured for motion in accordance with a predetermined number of degrees of freedom (DOF), and wherein the first set of actuators includes two actuators corresponding to at least one DOF.

7. A master-slave endoscopy system comprising:
an endoscope having a main body from which a flexible elongate shaft extends, the flexible elongate shaft spanning a length between a proximal end and a distal end thereof, the flexible elongate shaft having a set of channels disposed therein along its length into which a set of actuation assemblies are insertable, the plurality of channels including a first channel and a second channel;
a set of flexible robotically driven actuation assemblies carried by the set of channels, each robotically driven actuation assembly including:
a robotic arm having a robotically driven end effector coupled thereto; and
a plurality of tendons coupled to the robotic arm and configured for controlling motion of the robotic arm and its end effector in accordance with a predetermined number of degrees of freedom (DOF), wherein two tendons control each DOF of the robotic arm;
a set of actuators corresponding to each robotically driven actuation assembly, each actuator controllable by way of a set of input devices with which a surgeon can interact, each actuator configured for selectively applying torque to a tendon of its corresponding robotically driven actuation assembly in response to surgeon input directed to the set of input devices, wherein two actuators control each DOF of the robotic arm; and
a processing unit configured for performing a tendon pretensioning or retensioning procedure to automatically establish a level of tension in the plurality of tendons of each robotically driven actuation assembly by way of:
(a) applying torque to each actuator of the robotically driven actuation assembly in accordance with stored torque parameters associated with a representative tortuosity configuration that is expected to correspond to a tortuosity of a path along which the robotically driven actuation assembly is routed; or
(b) for each tendon of the robotically driven actuation assembly:
dynamically determining a torque transition point between a slack condition and a no-slack condition of the tendon; and
applying torque to an actuator corresponding to the tendon at a torque level defined by the torque transition point determined therefor.

8. The system of claim 7, wherein applying torque to each tendon of the robotically driven actuation assembly in accordance with stored torque parameters associated with the representative tortuosity configuration is performed outside of an operating theater prior to performance of an endoscopic procedure, or after insertion of each robotically driven actuation assembly into a channel of the flexible elongate shaft.

9. The system of claim 7, wherein dynamically determining for each tendon the torque transition point between the slack condition and the no-slack condition therefor occurs immediately prior to or during performance of an endoscopic procedure.

10. The system of claim 7, wherein dynamically determining for each tendon the torque transition point between the slack condition and the no-slack condition comprises:
measuring a tendon tension profile corresponding to the tendon; and
calculating a first and/or a second derivative of the tendon tension profile.

11. The system of claim 7, further comprising an instrument adapter corresponding to each robotically driven actuation assembly, the instrument adapter removably couplable to the set of actuators for selectively coupling the plurality of tendons robotically driven actuation assembly to the set of actuators, wherein the instrument adapter is configured for maintaining tension applied to each tendon of the robotically driven actuation assembly when decoupled from the set of actuators.

12. A master-slave endoscopy system comprising:
a set of robotically driven actuation assemblies, each robotically driven actuation assembly including:
a robotic arm having a robotically driven end effector coupled thereto; and
a plurality of tendons configured for controlling motion of the robotic arm and the end effector in accordance with a predetermined number of degrees of freedom (DOF); and
an instrument adapter corresponding to each robotically driven actuation assembly and coupled to the tendons thereof, the instrument adapter couplable to a set of mechanical elements for selectively coupling the plurality of tendons of the robotically driven actuation assembly to a set of actuators, the instrument adapter comprising:
a rotatable shaft corresponding to each tendon of the robotically driven actuation assembly, the rotatable shaft having a longitudinal axis relative to which the tendon is circumferentially wound; and
a first tension maintenance element and a second tension maintenance element corresponding to each rotatable shaft, wherein the first tension maintenance element is displaceable relative to the second tension maintenance element for selective engagement with and disengagement from the second ratchet element, and wherein the first tension maintenance element is configured for mating engagement with the second tension maintenance element when the instrument adapter is decoupled from the set of mechanical elements to prevent rotation of the shaft and thereby maintain a level of tension in the tendon.

13. The system of claim 12, wherein the instrument adapter further comprises a resilient biasing element that maintains the first tension maintenance element and the second tension maintenance element in an engaged state when the instrument adapter is decoupled from the set of mechanical elements, wherein the first and second tension maintenance elements each comprises one of a ratchet element and a friction plate.

14. The system of claim 13, wherein the resilient biasing element is displaceable relative to the shaft for disengaging the first tension maintenance element from the second tension maintenance element when the instrument adapter is coupled to the set of mechanical elements such that the shaft is rotatable.

15. (canceled)

16. The system of claim 12, wherein the set of actuators includes two actuators corresponding to at least one DOF, and wherein for each DOF the instrument adapter includes a first rotatable shaft relative to which a first tendon is circumferentially wound and a second rotatable shaft relative to which a second tendon is circumferentially wound for controlling motion of the robotic arm and end effector of the robotically driven actuation assembly.

17. A master-slave endoscopy system comprising:
an endoscope having a main body from which a flexible elongate shaft extends, the flexible elongate shaft spanning a length between a proximal end and a distal end thereof, the flexible elongate shaft having a set of channels disposed therein along its length into which a set of actuation assemblies are insertable, the plurality of channels including a first channel and a second channel;
a set of robotically driven actuation assemblies, each robotically driven actuation assembly including:
a robotic arm having a robotically driven end effector coupled thereto;
a plurality of tendons coupled to the robotic arm and configured for controlling motion of the robotic arm and the end effector in accordance with a predetermined number of degrees of freedom (DOF); and
an outer sleeve surrounding the plurality of tendons;
a first instrument adapter corresponding to each robotically driven actuation assembly and coupled to the tendons thereof, the first instrument adapter couplable to a set of mechanical elements for selectively coupling the plurality of tendons of the robotically driven actuation assembly to a set of robotic arm/end effector manipulation actuators; and
a translation mechanism configured for independently translating each robotically driven actuation assembly along a predetermined fraction of the length of the flexible elongate shaft to effectuate surge displacement of the robotically driven actuation assembly, the translation mechanism comprising one of:
(a) a collar carried by each outer sleeve of the set of robotically driven actuation assemblies; and
a translation unit comprising:
a receiver configured for matingly receiving an outer sleeve of a robotic ally driven actuation assembly; and
a linear actuator corresponding to each receiver and configured for selectively translating the receiver along the predetermined fraction of the flexible elongate shaft's length;
(b) a second instrument adapter to which each first instrument adapter is matingly engageable for coupling the tendons of the robotically driven actuation assembly corresponding to the first instrument adapter to the set of robotic arm/end effector manipulation actuators; and
a translation unit configured for carrying each first instrument adapter as well as a second instrument adapter matingly engageable therewith, and displacing each first instrument adapter and each second instrument adapter that are matingly engaged to effectuate surge displacement of individual robotically driven actuation assemblies along the predetermined fraction of the flexible elongate shaft's length; and
(c) a translation unit configured for displacing individual sets of robotic arm/end effector manipulation actuators and each first instrument adapter coupled thereto to effectuate surge displacement of individual robotically driven actuation assemblies along the predetermined fraction of the flexible elongate shaft's length.

18. The system of claim 17, wherein each second instrument adapter is coupled to the set of robotic arm/end effector manipulation actuators by a tether having a plurality of tendons therein.

19. The system of claim 17, further comprising a docking station to which a portion of the main body of the endoscope is detachably engageable, wherein the translation mechanism is carried by the docking station.

20. The system of claim 18, further comprising a patient side cart that carries the docking station.

21. The system of claim 17, further comprising a set of cradles carrying the translation mechanism, wherein each cradle of the set of cradles corresponds to an individual robotically driven actuation assembly, and each cradle of the set of cradles is coupled to a roll motion actuator configured for individually rotating the cradle and its corresponding robotically driven actuation assembly about a roll axis to provide roll motion to the robotic arm and end effector of the robotically driven actuation assembly.

22. The system of claim 21, further comprising a docking station to which a portion of the main body of the endoscope is detachably engageable, wherein the docking station carries the translation mechanism and the set of cradles.

23.-38. (canceled)
